(19) United States
(12) Patent Application Publication
(10) Pub. No.: US 2022/0125711 A1
(43) Pub. Date: Apr. 28, 2022

(54) CANNABINOID-CONTAINING ORAL THIN FILM FORMULATIONS

(71) Applicant: JDEM Properties, LLC, Skokie, IL (US)

(72) Inventors: Akhil Bajaj, Denver, CO (US); Skyler Webb, Denver, CO (US); Shehzad Hoosein, Chicago, IL (US); Andrew Rashkow, Skokie, IL (US)

(21) Appl. No.: 17/079,214

(22) Filed: Oct. 23, 2020

(51) Int. Cl. A61K 9/00; A61K 47/36; A61K 31/07; A61K 31/375; A61K 31/355; A61K 31/714; A61K 31/05; A61K 31/4415; A61K 36/82

(52) U.S. Cl. CPC A61K 9/006; A61K 47/36; A61K 31/07; A61K 31/375; A61K 36/82; A61K 31/714; A61K 31/05; A61K 31/4415; A61K 31/355

(57) ABSTRACT

The methods disclosed herein permit the synthesis of cannabinoid-containing oral thin film (OTF) strips that also contain caffeine or multiple additional active ingredients. Selection of film forming agents based on water usage coupled with low temperature mixing and low temperature drying may in various embodiments permit the incorporation of up to five additional ingredients into a cannabinoid containing OTF product.

### FIELD OF INVENTION

Formulations described in the present disclosure are designed for fast and efficient sublingual delivery of cannabinoids by use of a thin, flexible, oral thin film (OTF) strip which readily dissolves upon administration under the tongue. Optional variants incorporating additional active ingredients include formulations for sleep enhancement and energy enhancement.

### INCORPORATION BY REFERENCE

Each document cited herein is incorporated by reference in its entirety for all purposes.

### BACKGROUND

Fast dissolving oral thin films (OTFs) are a convenient alternative to other drug delivery methods and have been increasing in popularity in recent years. OTFs provide fast, accurate dosing of active ingredients in a safe, convenient, and efficacious format without the need for additional measuring, additional water, or additional equipment at the time of use by the consumer. Provided as individual strips, these films dissolve rapidly when placed in contact with a wet surface or environment, e.g. on or underneath the tongue or in the oral cavity. The matrix comprising the film dissolves in the aqueous environment of the oral cavity or sublingual space and active ingredients cross the oral mucosa and directly enter the systemic circulation, avoiding first pass metabolism and degradation in the gastrointestinal tract. The epithelial cells of the sublingual mucosa comprise a relatively thin membrane and are proximal to large veins, providing high bioavailability of active ingredients delivered by sublingual OTFs. OTF formulations generally contain a strip-forming matrix component, plasticizers, bulking agents, active ingredients, sweeteners, flavoring agents, bitter blockers, coloring agents, stabilizing agents, thickening agents, or permeation enhancers, among other possible ingredients.

OTF formulations may comprise emulsions, wherein water-soluble hydrophilic active ingredients are dissolved in the aqueous phase and lipophilic active ingredients are dissolved in the oil phase. OTF formulations comprising emulsions may optionally include surfactants and co-surfactants to promote emulsification of the oil phase and lipophilic active ingredients in the aqueous phase of the OTF formulation.

Cannabis plants produce phytocannabinoids, a diverse class of organic compounds that act on cannabinoid receptors in human physiology. A subset of phytocannabinoids have various physical and mental effects when extracted from the plant and consumed by a human subject and may be used for both therapeutic and recreational purposes.

There are many routes for administering plant-derived cannabinoids in humans, including smoking, vaporizing, oral ingestion, transdermal patch, and intravenous injection, and each of these delivery methods may have various advantages and disadvantages. Notably, many of these administration methods of cannabinoids exhibit unpredictable time to onset, duration, and intensity of the desired physical and mental effects due to variability in dosing, bioavailability, and metabolism. Oral administration of cannabinoids by delivery of active ingredients to the oral or sublingual mucosa via OTF formulations may address these disadvantages.

OTF formulations have been used to orally deliver various therapeutic active ingredients including nicotine, loratadine, flurazepam, oxycodone, dextromethorphan HCl, among others. However, a well-known challenge for incorporating active ingredients into OTF formulations is the capacity of the strip for loading active ingredients. One noted disadvantage of OTF formulations compared to other oral method of delivery, e.g. capsules, liquigels, or orally dissolvable tablets, is that high doses of active ingredients cannot be incorporated into the strip. Previously disclosed OTF formulations generally accommodate only about 7-15% (w/w) of active ingredient. US 2003/0206942 to Kulkarni et al., for example, achieved in his examples an active ingredient load of between 7 and 8% (w/w). WO 2020/014776 to Macphail (“Macphail”) achieved active ingredient loads of 12-15%, as shown in his Table 5.

OTF formulations have also been limited in the number of different active ingredients that may be incorporated into the strip. Including an efficacious dose of one particular active ingredient may limit the number or amount of additional ingredients that may be incorporated into the aqueous phase or the oil phase of the emulsion that comprises the strip due to solubility limitations of each phase. There are several challenges that have prevented the incorporation of many additional active ingredients in a single cannabinoid-containing OTF strip.

First, solute-solute interactions usually impede the solubility of one or both solutes, which substantially prevents the incorporation of the solute into a dried OTF film and impairs the homogeneity of the solute in the dried OTF film. This phenomenon can be observed, for instance, when a protein is “salted out” of a water solution. Adding a salt such as ammonium sulfate reduces the solubility of the protein in water, causing it to precipitate or crystallize out. In like fashion, adding one active ingredient often has an unpredictable and adverse effect on the solubility of the other active ingredient. The severity of the effect is difficult to predict and depends on many factors including the polarity or ionic nature of the solutes and solvent, pressure, temperature and other factors. Where three or more solvents or active ingredients are used, the solution behavior is exponentially more unpredictable and one or more of the active ingredients typically precipitates out of solution during the preparation process or is incorporated only heterogeneously into the OTF product. Such active ingredient heterogeneity substantially reduces sublingual solubility and uptake.

Second, crystallization has detrimental effects on the mechanical and mucoadhesive properties of the oral thin strip formulations, on active ingredient content uniformity throughout the strip, and on active ingredient release and associated kinetics as the thin strip dissolves and active ingredients cross the sublingual epithelium. Crystallization of active ingredients may also result inconsistent thickness of the OTF strip. Crystallization is generally due to use of multiple solutes (active ingredients) and crystallization of an active ingredient substantially reduces homogeneity of the OTF strip and the effectiveness and uptake of the active ingredient. Multiple solutes interact which one another in various and unpredictable ways, especially in the case of polar or ionic solutions. Solute-solute complexes may be formed, which in turn provide nucleation sites for crystallization. The presence of suitable nucleation sites within a solution greatly increases the chances of unwanted crystallization and, correspondingly, degradation of the amount and homogeneity of the active ingredients in the resulting OTF product. The crystallization of active ingredient substantially reduces total uptake and rate of uptake sublingually. The uptake rate and amount is a function of the homogeneity of the film and the rate at which the active ingredient is brought into aqueous solution at the cellular wall. Crystallization inhibits dissolution of the OTF composition sublingually and thus inhibits uptake and absorption through the endothelium.

Third, inclusion of multiple active ingredients is particularly challenging when one of the active ingredients is a cannabinoid. Cannabinoid oils are highly viscous and notoriously difficult to homogenize in aqueous phases. As explained in WO2016147186A1 to Sinai et al. cannabinoids are highly lipophilic, essentially water insoluble, and difficult to solubilize for inclusion in pharmaceutical formulations.

Recent patent publications reflect these challenges to the incorporation of multiple active ingredients, especially caffeine, in a cannabinoid OTF product. U.S. Pat. No. 10,265,362 to Schaneville (“Schaneville”) discloses a mucosally dissolvable film containing a matrix and a cannabinoid. Schaneville broadly teaches cannabinoid-containing emulsion compositions comprising “one or more” active ingredients, matrix components, and liquid ingredients that may be mixed, dried, and cut into individual strips. However, Schaneville discloses only the successful inclusion of a single active ingredient in the OTF strip. Shaneville's Table 1 teaches an OTF formulation including as an active ingredient only one cannabinoid in the amount of 12-70 mg active per strip. Shaneville does not disclose any specific formulations with multiple active ingredients.

WO 2020/014776 to Macphail (“Macphail”) also broadly teaches an oral dissolvable film composition comprising a cannabinoid and a film forming agent, as well as a method of manufacturing the formulation. In the disclosed examples, Macphail teaches stirring the liquid components of the formulation while heating at 70° C. (158° F.) and successively adding additional components of the formulation (see Macphail at p. 27). The matrix-forming component of Macphail's illustrative examples is pullulan (see Macphail at p. 25; Table 1). Further, Macphail teaches drying the OTF formulation in a convection style oven set to 70° C. (158° F.) with an air temperature of 100° C. (212° F.). Macphail's technique succeeded in incorporating a cannabinoid and second active ingredient, melatonin or methylcobalamin, onto an OTF film.

In yet another previous disclosure of cannabinoids delivered orally by OTF formulations, US 2019/0269649 to Allen (“Allen”) teaches an oral dissolvable film composition comprising two active ingredients derived from botanical extracts, specifically cannabinoids. Allen teaches mixing a lipid, emulsifier, and solvent at an elevated temperature of about 54.4-60° C. (130-140° F.) to produce a first uniform mixture and adding an active ingredient while mixing at room temperature (about 70° F.) to produce a thickened second mixture. Allen further teaches condensing and drying of the mixed OTF formulation in a convection oven at 70° C. (158° F.). In the disclosed examples, Allen teaches a formulation that contains a cannabinoid and a kavalactone, which is a lactone compound derived from the kava shrub that may have various psychotropic effects. Allen, like MacPhail, was able to incorporate one additional active ingredient into a cannabinoid OTF product.

Allen specifically discusses one of the challenges noted above. Allen teaches that “[w]ith some active ingredients (e.g. caffeine), upon cooling, the active ingredient will fall out of solution (i.e. precipitate), due to poor solubility issues.” Precipitation of active ingredients out of solution remains a well-known problem in the OTF field and this problem is particularly acute in formulations involving cannabinoids and/or caffeine, which are inherently difficult to solubilize in an aqueous medium. MacPhail teaches that a “crystallization inhibitor” may be added to the formulation but does not disclose any suitable crystallization inhibitors. Indeed, no “crystallization inhibitors” for caffeine are currently known in the art to the best of Applicant's knowledge.

US 2020/0069639 to Ghalili et al. (“Ghalili”) also teaches an orally dissolvable thin film or strip comprising a film forming agent, at least one cannabinoid, and several additional active ingredients, e.g., menthol, caffeine, or various vitamins. However, the teachings of Ghalili are incomplete, internally inconsistent, and irreproducible by a skilled artisan for the reasons discussed below.

First, the illustrative examples provided by Ghalili lack teachings necessary to produce the claimed invention and are likely prophetic or fictional. Considering Example 1, Ghalili teaches as a component of a strip formulation “strip material (glycerin, algin, pullulan, propylene glycol, corn starch, flavorings (peppermint oil, spearmint), coloring (tartrazine), sweetener (sucrose) and polysorbate 80) 30.49 wt % (32.75 mg).” The components of the strip material are provided but neither their percent by weight relative to each other nor relative to the total strip formulation are provided. This teaching provides the percent by weight of the total formulation that the strip material comprises, however, does not teach weights or proportions for each individual strip material component. Film-forming agents, e.g., glycerin, algin, pullulan, each have specific water usage rates, viscosities, and unique film-forming properties, and their relative proportions affect the properties of the final orally dissolvable thin strip.

TABLE 1

| | Example 1 | Example 2 | Example 3 |
|---|---|---|---|
| menthol (mg) | 16 | 190.8 | 285 |
| caffeine (mg) | 10 | 300 | 45 |
| hemp oil (mg) | 2 | 30 | 45 |
| vitamin B2 (mg) | 5 | 300 | 300 |
| vitamin B3 (mg) | 50 | 100 | 100 |
| vitamin B5 (mg) | 50 | 20 | 20 |
| vitamin B6 (mg) | 2.5 | 50 | 50 |
| vitamin B7 (mg) | 30 | 10 | 10 |
| strip material (mg) | 32.75 | 32.75 | 32.75 |
| stated total (mg) | 107.44 | 1132.8 | 1716 |
| actual total (mg) | 198.25 | 1033.55 | 887.75 |

Second, as shown in Table 1, above, Ghalili's examples are internally inconsistent with respect to the total weights and relative amounts of the components of the formulations. Example 1 teaches a strip with a total weight of 107.44 mg but the sum of the weight of all the components listed in Example 1 is 198.25 mg. Example 2 states a total weight of 1132.8 mg, however, the sum of the weight of all the components listed in Example 2 is 1033.6 mg. Example 3 states a total weight of 1716 mg, yet the sum of the weight of all the components listed in Example 3 is 887.8 mg. Thus, in each example the recited total weight of ingredients in the formulation is incongruent with the actual sum of the recited weights of each component. However, it is not apparent whether the stated total weight, the actual total weight of listed components, or neither is correct. For Example 1, the sum of the weights of listed components is larger than the stated total weight. For Examples 2 and 3 the sum of the weights of the listed components is less than the stated total weight.

Finally, Ghalili teaches that “[a]ny standard manufacturing procedure known in the art may be used to manufacture the tape, film or strip or segments thereof of the present disclosure.” Ghalili goes on to describe basic steps of mixing, casting, and drying, without any additional details. Variables including mixing speeds, drying time, and drying temperature, among other variables that may affect the properties and viability of an orally dissolvable thin strip, are not described in the specification or the illustrative examples.

In light of the foregoing, the illustrative examples of Ghalili are likely to be either prophetic or fictional. They are incomplete, internally inconsistent, and are not reproducible by a person of skill in the art.

To date, the foregoing challenges have prevented the development of a cannabinoid OTF formulation with more than one additional active ingredient. There is thus a long felt and unmet need for orally consumable cannabinoid-containing OTF formulations that accommodate a plurality of additional active ingredients, i.e., in addition to the cannabinoid.

Similarly, due to the particular difficulties associated with caffeine, it has not been possible to prepare a cannabinoid-containing OTF strip that also includes a therapeutically effective amount of caffeine. There is likewise a long felt and unmet need for such an OTF formulation.

### SUMMARY OF ILLUSTRATIVE EMBODIMENTS

The methods disclosed herein permit the synthesis of cannabinoid-containing OTF strips that also contain caffeine or multiple additional water soluble or oil soluble active ingredients. While not wanting to be bound to any particular theory, the choice of matrix components included in the OTF formulation may substantially influence the number and amount of active ingredients that may be included in the OTF formulation and final dried strip. For example, pullulan, sodium alginate, xanthan gum, and waxy maize starch each “consume” varying amounts of water when solubilized in the aqueous phase of the OTF emulsions, thereby influencing the amount of water that is available to solubilize additional active ingredients or high quantities of additional active ingredients. The careful selection of matrix components included in the OTF formulation based on water consumption is believed to substantially influence how many active ingredients may be included in the OTF formulation while avoiding precipitation and recrystallization.

Further, Applicant observes that the use of high temperature preparation methods may aid in solubilizing additional active ingredients but may disadvantageously degrade one or more temperature-sensitive active ingredients during preparation. Applicant further observes that high temperature preparation methods may also require that high temperature drying must also be used, else the film-forming composition will rapidly cool and the additional ingredients will precipitate out of solution or, at a minimum, partially coalesce and substantially impair the texture and homogeneity of the resulting film. While still not wanting to be bound by a particular theory, the use of high temperature drying is currently believed to promote crystallization of the active ingredients. Rapid changes in the matrix brought about by rapid drying may cause the proliferation of nucleation sites and the growth of crystals of active ingredients. The crystallization may cause the active ingredient to precipitate out of the film forming solution and is likely to substantially degrade the homogeneity and texture of the resulting OTF product. Crystallization also impedes sublingual uptake or absorption of the active ingredients, as discussed above.

Low temperature drying has previously been considered undesirable in many respects, particularly in that it substantially prolongs the drying process and can impede efficient production of the OTF product. However, Applicant observes that use of low temperature drying may avoid the degradation of temperature-sensitive active ingredients and mitigate the recrystallization of active ingredients in the final dried OTF strip.

Low temperature preparation has previously been considered less desirable for incorporation of some active ingredients because solubility of some active ingredients is substantially impaired at lower temperatures. However, Applicant observes that the choice of appropriate film-forming agents may allow for lower temperature preparation and solubilization of active ingredients while providing the substantial advantage of avoiding the degradation of temperature-sensitive active ingredients. Thus, if the challenges of low temperature preparation, i.e., solubilization of active ingredients, can be overcome, there exists an opportunity to utilize low temperature preparation which may avoid degradation of temperature-sensitive active ingredients and recrystallization of active ingredients in the final dried cannabinoid OTF product.

Applicant has found that, unexpectedly, careful selection of film-forming agents that underly the matrix of the final dried OTF strip may permit the successful navigation of these challenges and creation of an OTF product having multiple active ingredients in addition to a cannabinoid. The film forming matrix components may be chosen, among other things, based substantially on their water-holding capacity (WHC), which affects the amount of free water available to solubilize the water-soluble active ingredients. Further, applicant has also found that OTF products utilizing the chosen film forming matrix components are able to retain oil-based extracts and oil-based active ingredients within the OTF matrix. Thus, film-forming agents may also be chosen based substantially on their oil-holding capacity (OHC). In a preferred embodiment, modified waxy maize starch is used, among other film-forming agents, due to its relatively high oil-holding capacity. This film forming matrix may be successfully processed at low temperatures while solubilizing and suspending multiple active ingredients in addition to the oil-soluble cannabinoid, which is itself quite difficult to emulsify and retain within the matrix of the final dried OTF strip. Still further, film-forming agents may be chosen based on their viscosity, which influences the quantity of film-forming agent required to form a stable matrix in the final dried OTF strip. While still not wishing to be bound to a particular theory, the careful selection of film-forming agents based on water-holding capacity, oil-holding capacity, and viscosity is believed to permit the use of low temperature preparation and low temperature drying. The low temperature drying is believed to prevent the formation of nucleation sites within the film forming matrix and that, in turn, may permit in many processes the successful formation of a final dried OTF product having acceptable homogeneity, texture, and solubility on the tongue of a user. Additionally, while still not wishing to be bound to a particular theory, the careful selection of matrix is believed to permit the tunability of the efficacies of each multiple active ingredients without altering the matrix contents and the usage rates, whereas typical OTF formulations require modifications to their matrix contents and/or the usage rates in order to change the efficacies of an active ingredient.

The methods disclosed herein achieve, for the first time, an OTF product having a plurality of active ingredients in addition to the cannabinoid. The methods also permit, for the first time, the preparation of an OTF product including both a cannabinoid and caffeine. As noted above, caffeine is particularly susceptible to crystallization. Heretofore no OTF film known to Applicant has successfully incorporated both a cannabinoid and caffeine. The methods additionally permit the preparation of an OTF producing including combinations of active ingredients including cannabinoid and valerian root extract, cannabinoid and lavender extract, or cannabinoid, valerian root extract, and lavender extract. The present invention achieves these long-felt needs.

Applicant notes that the compositions and methods disclosed herein have the further substantial benefit that the active ingredients, especially the cannabinoid, are spared from the degradation caused by exposure to elevated temperatures. Previously known high temperature mixing techniques likely caused substantial degradation of the active ingredients and thus impaired the effectiveness of the resulting OTF product. The Applicant's preferred techniques avoid this temperature-related degradation of active ingredients and thus produce OTF products of enhanced efficacy.

In various embodiments, a method of manufacturing an oral dissolvable film comprises a therapeutically effective amount of a first active ingredient that is a cannabinoid, a therapeutically effective amount of a second active ingredient different from the first active ingredient, a therapeutically effective amount of a third active ingredient different from the first and second active ingredients, a therapeutically effective amount of a fourth active ingredient different from the first, second and third active ingredients, and further, combining the first active ingredient, second active ingredient, third active ingredient, fourth active ingredient, and a film forming agent form a stable homogenous dispersion; casting the stable dispersion on a substrate into a sheet having a thickness less than about 800 μm; drying the dispersion on the substrate at one or more temperatures less than 50° C. for a total duration of at least one hour to form the oral dissolvable thin film; and forming the dried film into strips adapted for use sublingually. In additional embodiments, the oral dissolvable film may further include a therapeutically effective amount of a fifth active ingredient different from the first, second and third and fourth active ingredients. In additional embodiments, the oral dissolvable film may further include a therapeutically effective amount of a sixth active ingredient different from the first, second and third, fourth and fifth active ingredients. In such embodiments, it may be the case that none of the third active ingredient or fourth active ingredient comprises a cannabinoid.

In another embodiment, the film forming agent is one or more of modified waxy maize starch, xanthan gum, or sodium alginate. In such embodiments, the film forming agent has a water usage substantially less than that of pullulan. In additional embodiments, the film forming agent may comprise less than 30% of the oral dissolvable thin film by weight. In additional embodiments, the active ingredients comprise more than 40% of the oral dissolvable thin film by weight. In additional embodiments, the ratio of active ingredients to film forming agent in the oral dissolvable thin film is at least 1.3:1 by weight. In some embodiments, the first active ingredient is one or more of THC, THCA, or CBD. In additional embodiments, the second active ingredient comprises valerian root extract, lavender extract, L-theanine, or L-tryptophan. In some embodiments, the forming step comprises laser cutting the dried film into strips each having a mass less than 120 mg and thickness less than about 500 μm. Some embodiments comprise an oral dissolvable film manufactured utilizing the methods described above. Some embodiments may have a percentage relative standard deviation of potency concentration of the at least one active ingredient in a batch of said oral dissolvable film composition of less than 1%.

In other embodiments, a method of manufacturing an oral dissolvable film comprises a therapeutically effective amount of a first active ingredient that is a cannabinoid, a therapeutically effective amount of a second active ingredient that comprises caffeine, a therapeutically effective amount of a third active ingredient different from the first and second active ingredients, and a therapeutically effective amount of a fourth active ingredient different from the first, second and third active ingredients, wherein the method comprises combining the first active ingredient, second active ingredient, third active ingredient, fourth active ingredient, and a film forming agent to form a stable homogenous dispersion; casting the stable dispersion on a substrate into a sheet having a thickness less than about 800 μm; drying the dispersion on the substrate at one or more temperatures less than 50° C. for a total duration of at least one hour to form the oral dissolvable thin film; and forming the dried film into strips adapted for use sublingually.

In additional embodiments, the oral dissolvable film further includes a therapeutically effective amount of a fifth active ingredient different from the first, second and third and fourth active ingredients. In other embodiments, the oral dissolvable film further includes a therapeutically effective amount of a sixth active ingredient different from the first, second and third, fourth and fifth active ingredients. In additional embodiments, the oral dissolvable film further includes a therapeutically effective amount of a seventh active ingredient different from the first, second and third, fourth and fifth and sixth active ingredients. In some embodiments, none of the third active ingredient, fourth active ingredient, fifth active ingredient, sixth active ingredient, or seventh active ingredient comprises a cannabinoid.

In additional embodiments, the film forming agent is one or more of modified waxy maize starch, xanthan gum, or sodium alginate. In other embodiments, the film forming agent has a gelling capability substantially higher than that of pullulan at a lower usage rate. In some embodiments, the film forming agent comprises less than 30% of the oral dissolvable thin film by weight. In other embodiments, the active ingredients comprise more than 40% of the oral dissolvable thin film by weight. In additional embodiments, the ratio of active ingredients to film forming agent in the oral dissolvable thin film is at least 1.3:1 by weight. In additional embodiments, the first active ingredient is one or more of THC, THCA, or CBD. In other embodiments, the third active ingredient comprises one of green tea extract, yerba mate extract, or vitamin B complex. In other embodiments, the fourth active ingredient comprises one of terpenes, vitamin A, vitamin C, or vitamin E. In additional embodiments, the methods include solubilizing one or more water-soluble active ingredients and one or more oil-soluble active ingredients in addition to the one or more cannabinoid-containing active ingredient.

In additional embodiments, the forming comprises laser cutting the dried film into strips each having a mass less than 120 mg and thickness less than about 500 μm. Other embodiments comprise an oral dissolvable film manufactured utilizing the methods describes above.

The foregoing general description of the illustrative implementations and the following detailed description thereof are merely exemplary aspects of the teachings of this disclosure and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale.

Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1 is a flow chart diagram showing the general steps of making a cannabinoid containing sublingual film.

FIG. 2A is a schematic representing the film casting process used to produce the OTF strips disclosed herein, wherein a liquid composition has been loaded into a film casting knife.

FIG. 2B is a schematic representing the film casting process used to produce the OTF strips disclosed herein, wherein the film casting knife has been operated to produce a thin film of user-defined thickness.

FIG. 3 is a photograph of final semi-dehydrated OTF strips produced by the methods disclosed herein.

### DETAILED DESCRIPTION OF ILLUSTRATIVE EMBODIMENTS

Cannabinoid-containing OTF formulations as described herein can be safely and predictably administered through the use of the sublingual administration of a thin layer of a partially dehydrated emulsion, which contains a measured dose of a cannabinoid. In some embodiments, this formulation and administration method allows for faster absorption, faster onset, as well as a more predictable effect than many other administration formulations currently available. In some embodiments, the OTF formulations may include a plurality of active ingredients, for example, up to 2, 3, 4, 5, 6, 7, 8, 9, or 10 active ingredients.

The description set forth below in connection with the appended drawings is intended to be a description of various illustrative embodiments of the disclosed subject matter. Specific features and functionalities are described in connection with each illustrative embodiment; however, it will be apparent to those skilled in the art that the disclosed embodiments may be practiced without each of those specific features and functionalities.

All references cited herein are hereby incorporated by reference in their entirety for all purposes.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

The term “gradually” as used herein refers to a change that occurs slowly, or by degrees, or “little by little”, rather than all at one time. The change can be, for example, a change in temperature, a change in mixing speed, or even the slow addition of one set of ingredients to another.

In certain embodiments, the term “gradually” regarding a temperature change, refers to a slow change in temperature (increase or decrease) from one temperature to another. For example, a gradual increase from a temperature x to a temperature y can occur, for example, in about 5 minutes, 10 minutes, 15 minutes, 20 minutes, 30 minutes, 45 minutes, to about 1 hour.

In certain embodiments, the term “gradually”, when referring to mixing speed, refers to a gradual change in mixing speed from one speed to another, where the change takes place, for example, from about 5 minutes, about 5 minutes, 10 minutes, 15 minutes, 20 minutes, 30 minutes, 45 minutes, to about 1 hour.

In certain embodiments, the term “gradually” refers to timing of addition of one mixture to another, such as an oil phase to an aqueous phase. For example, the gradual addition of a pre-mixed oil phase can occur over a time range of about 3 minutes, 5 minutes, 10 minutes, 20 minutes, 30 minutes, to about 45 minutes. In an embodiment, the aqueous and oil phase ingredients are well mixed at a lower speed before the transition to high speed mixing.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

The term “cannabinoid oil” as used herein refers to an extract from Cannabis plants. Cannabis is a genus the eukaryotic plant family Cannabaceae. Three common species in this genus are Cannabis sativa, Cannabis indica, and Cannabis ruderalis.

Cannabinoids are a diverse group of chemical compounds that act on cannabinoid receptors, e.g. cannabinoid receptor type 1 (CB1) and cannabinoid receptor type 2 (CB2). Numerous cannabinoids can be used in the embodiments described herein. Suitable cannabinoids include, for example, cannabinol, cannabidiol (CBD), tetrahydrocannabinol (THC), tetrahydrocannabinolic acid (THCA), Δ9-tetrahydrocannabinol, cannabidiolic acid (CBDA), Δ8-tetrahydrocannabinol, 11-hydroxy-tetrahydrocannabinol, 11-hydroxy-Δ9-tetrahydrocannabinol, levonantradol, Δ11-tetrahydrocannabinol, cannabidivarin (CBDV), tetrahydrocannabivarin, cannabidivarinic acid (CBDVA), cannabitriol, dronabinol, amandamide, nabilone, and combinations thereof.

CBD can include, for example, delta-1-cannabidiol, delta-2-cannabidiol, delta-3-cannabidiol, delta-3,7-cannabidiol, delta-4-cannabidiol, delta-5-cannabidiol, delta-6-cannabidiol, and combinations thereof.

In an embodiment, the cannabinoid oil can be decarboxylated. Decarboxylation is a chemical reaction that releases carbon dioxide and generates neutral and biologically active cannabinoids. The decarboxylation process can occur, for example, by heating or by solvent extraction.

The concentration of the cannabinoid in the formulation can be about 1%, 4%, 6%, 10%, 15%, 20%, 25%, 30%, 35%, 40%, 50% 60%, 70%, 80%, or about 90% and the cannabinoid extract may be one of a solvent extract, a distillate, an isolate, among other methods of extracting cannabinoid compounds from their plant sources.

The methods shown herein allow for a large range of cannabinoid in the final OTF product. For example, in an embodiment, each final individual OTF strip may contain from about 1 mg, 5 mg, 10 mg, 20 mg, 50 mg, 100 mg, or more of cannabinoid. The final semi-dehydrated OTF strips may each have an individual total weight of about 1, 2, 3, 4, 5, 10, 15, 20, 25, 30, 35, 40, 45, 50, 55, 60, 65, 70, 75, 80, 85, 90, 95, 100, 105, 110, 115, 120, 125, 130, 135, 140, 145, or 150 milligrams. In another embodiment, the final semi-dehydrated OTF strips may each have an individual total weight of about 200, 250, 300, 350, 400, 450, or 500 milligrams.

The cannabinoid used in various embodiments described herein can be a pure form of cannabinoid isolate. The cannabinoid can also be in the form of a purified full spectrum Cannabis oil extract. The cannabinoid can also be present as a mixture of compounds extracted from the plant. The cannabinoid oil can be extracted, for example from plants of the genus Cannabis, using, for example, methods such as organic solvent extraction, steam distillation, microwave-assisted solvent extraction, cryo-mechanical methods, supercritical fluid extraction, water extraction, and other extraction methods. The cannabinoid oil can also be mixed with other oils.

In another embodiment the cannabinoid oil can be extracted from microorganisms, such as genetically modified algae, bacteria, or fungi. In yet another embodiment, the cannabinoid oil is prepared synthetically.

Embodiments of the present disclosure utilize an edible, easily dissolvable, OTF formulations yielding a partially dehydrated strip to sublingually administer at least one cannabinoid, as well as other active ingredients. In an embodiment, the oral thin strip formulations comprise 1, 2, 3, 4, 5, 6, 7, 8, 9 or up to 10 active ingredients in addition to the one or more cannabinoids. In another embodiment, the oral thin strip formations comprise one or more water-soluble active ingredients and one or more oil-soluble active ingredients in addition to the one or more cannabinoids.

In an embodiment of the present disclosure, the oral thin strip formulation is prepared by mixing an aqueous phase (containing a mixture of various hydrophilic components) with an oil phase (containing various lipophilic components including a cannabinoid) to form an oil in water microemulsion. This allows for the cannabinoid to dissolve in the carrier oil, while a polar phase will also be present that can assist in the rapid dissolution of the formulation in the oral cavity when the OTF strip comes in contact with the aqueous environment of the sublingual oral mucosa.

An emulsion is a mixture of two or more liquids that are normally immiscible. Emulsions typically do not form spontaneously, as they are inherently unstable. Industrial processes may utilize such means as stirring, shaking, blending, ultrasound, or homogenization in order to form a more stable emulsion.

The term “microemulsion,” as used herein, describes an isotropic thermodynamically stable mixture of two immiscible fluids incorporating small particle sizes as compared to conventional emulsions. An oil-in-water (o/w) microemulsion is a dispersion of spheroid particles comprised of oil, surfactant, and possibly a co-surfactant, dispersed within an aqueous continuous phase.

In an embodiment, the composition is present as an emulsion, such as a microemulsion, having a hydrophobic phase (or oil phase) and a hydrophilic phase (or aqueous phase). The hydrophobic cannabinoids are dissolved into the oil phase, and the oil phase is incorporated into the aqueous phase. Thus, a nonpolar or “oil phase” is prepared, containing various lipoic moieties, including a cannabinoid extract. A polar phase (or “aqueous phase”) is separately prepared, which can contain polar ingredients such as water, other polar solvents, a surfactant, an emulsifier, a plasticizer, and a bulking agent. Flavoring agents, other terpenoids, coloring agents, and the like can be added to either the nonpolar or polar phase, as desired. Other active ingredients can also be added to either the oil phase or the aqueous phase.

In a preferred embodiment, the oil phase and the aqueous phase of the emulsion are first separately, gradually mixed at approximately room temperature, i.e. at about 25° C. Additional ingredients, such as emulsifiers and thickeners, can be added during this mixing phase. In an embodiment, additional ingredients are added in a specific order, and mixed thoroughly prior to addition of the next ingredient. Emulsifiers (for either the oil phase and/or the aqueous phase) can be added and mixed in during this mixing step. In an embodiment, the emulsifier is added to the oil phase or the aqueous phase at approximately room temperature, i.e. at about 25° C. The mixed oil phase can be gradually added to the mixed polar phase at approximately room temperature, i.e. at about 25° C.

In an embodiment of the present disclosure, the nonpolar phase, containing the carrier oils and the cannabinoid(s), is prepared by mixing at approximately room temperature, i.e. at about 25° C.

In another embodiment, the oil phase and the aqueous phase of the emulsion are first separately, gradually heated and mixed to a temperature range of about 55° C. to about 65° C. Additional ingredients, such as emulsifiers and thickeners, can be added during this warming phase. In an embodiment, additional ingredients are added in a specific order, and mixed thoroughly prior to addition of the next ingredient. Emulsifiers (for either the oil phase and/or the aqueous phase) can be added and mixed in during this warming step, while the temperature of the mixture gradually increasing. In an embodiment, the emulsifier is added to the oil phase or the aqueous phase at about 30° C., 35° C., 40° C., 45° C., 50° C., 55° C., 60° C., to about 65° C. The warmed oil phase can be gradually added to the warmed polar phase.

In an embodiment of the present disclosure, the nonpolar phase, containing the carrier oils and the cannabinoid(s), is prepared by mixing and heating up to a temperature range of from about 55° C. to about 65° C. For example, the mixture can be mixed at from about 55° C., 57° C., 60° C., 63° C., to about 65° C.

The utilization of a carrier oil for creating an oil extract micro-emulsion has many advantages. As a first point, decarboxylated Cannabis extract alone is often highly viscous and thus difficult to process. Dissolution in a medium chain triglyceride carrier oil can reduce its viscosity, allowing it to be easily mixed with the polar phase. Further, using Cannabis extract without a carrier oil when forming an emulsion can lead to coalescence of the emulsion, leading to a lowered stability of the emulsion.

Various oil types can be used, for example, various monoglycerides, diglycerides, or triglycerides. In an embodiment, the oil is a medium chain triglyceride (MCT). MCTs contain short and medium chain fatty acids (<12 carbons) and can serve as effective carrier oils for a microemulsion. MCT oil may comprise a mixture of molecules with fatty acid groups including caproic acid (C6), caprylic acid (C8), capric acid (C10) or lauric acid (C12). In an embodiment, the fatty acid group of the medium chain triglyceride has from about 6, 8, 10, or 12 carbons. The MCT oil can also be a mixture of triglycerides with fatty acid groups of various carbon lengths. In an embodiment the MCT formulation oil phase contains at least 95% caproic acid triglycerides. In another embodiment the MCT formulation oil phase contains at least 95% caprylic acid triglycerides. In another embodiment, the MCT formulation oil phase contains at least 95% capric acid triglycerides. In another embodiment the MCT formulation oil phase contains at least 95% lauric acid triglycerides.

The oil phase can be present at various percentages in the formulation. Thus, in an embodiment, the total oil in the formulation is about 1%, 2%, 3%, 4%, 5%, 7%, 10%, 13%, 15%, 17%, 20%, 22%, 25% or more of the formulation, on a weight to weight (w/w) basis. In an embodiment, there is a high load of oil such as, for example, greater than about 20%. In another embodiment, there is a low load of oil in the formulation, such as, for example, less than about 7%.

In an embodiment, the microemulsion formulations may be loaded with 10% on a weight to weight (w/w) basis of a cannabinoid oil having about 80% THC. In another embodiment, a cannabinoid oil may be present in the microemulsion formulations as about 5%, 10%, 15%, 20%, or more by weight (w/w).

In other embodiments, a low load of cannabinoid is desired. Accordingly, a cannabinoid oil can be present in the formulation from about 0.1%, 0.5%, to about 0.75%, 1%, 1.5%, 2%, 2.5%, 4%, or about 5% by weight of the formulation.

In an embodiment, the ratio of the oil to the emulsifier can be from about 2:1, 2.5:1, 3:1, 3.5:1, 4:1, 4.5:1, 5:1, 5.5:1, or 6:1, on a weight to weight basis. In an embodiment, the method and compositions provide for a lower amount of emulsifier needed in comparison to the amount of oil present. Lowering the amount of emulsifier needed can decrease the bitter or soapy taste of the formulation.

In various embodiments disclosed herein, MCTs contribute to the decreased onset time of desired physical and mental effects. While not wishing to be bound by theory, this is anticipated to be due to rapid absorption vasculature of the sublingual oral mucosa, mediated in part by the small particle sizes of emulsified cannabinoid-bearing oil phase in the formulations disclosed herein. The highly vascularized sublingual mucosa delivers active ingredients to the systemic circulation, bypassing first-pass metabolism or degradation in the gastrointestinal tract.

Medium chain triglycerides (MCTs) may be a caprylic acid triglyceride (C8). It is made using glycerol from vegetable oil sources and medium-chain fatty acids from coconut and palm kernel oils. MCTs are considered to be a healthier choice due to their efficient metabolism of burning fat to energy. MCTs may be, for example, about 95% C8, 96% C8, 97% C8, 98% C8, 99% C8, or more.

Additional exemplary oils include vegetable oils, almond oil, canola oil, coconut oil, cottonseed oil, soybean oil, safflower oil, sunflower oil, castor oil, corn oil, grapeseed oil, olive oil, palm oil, peanut oil, sesame oil, rapeseed oil, peppermint oil, poppy seed oil, palm kernel oil, walnut oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl monolinoleate, glyceryl palmitate, glyceryl palmitostearate, glyceryl stearate, polyglyceryl 10-oleate, polyglyceryl 3-oleate, polyglyceryl 4-oleate, polyglyceryl 10-tetralinoleate, caprylyic/capric glycerides and combinations thereof.

In an embodiment, there is a high load of THC (or other desired cannabinoid compound) in the oil phase. For example, formulations of certain embodiments can be loaded with 10% of an oil having about 80% THC (w/w). In other embodiments, a low load of cannabinoid is desired. Accordingly, a cannabinoid can be present in the formulation from about 0.1%, 0.5%, 0.75%, 1%, 1.5%, 2%, 2.5%, 4%, 5%, 7.5%, to about 10% (w/w).

In an embodiment, an aqueous phase, containing at least one polar solvent, is prepared. Although it is termed the “aqueous phase” or “polar phase”, this phase can contain polar solvents, non-polar solvents, and polyols in addition to or instead of water. The at least one aqueous phase solvent can be, for example, water, sorbitol, glycerol, propylene glycol, coconut water, glycerin, glucose, sucrose, sodium phosphate, methanol, ethanol, isopropyl alcohol, butanol, pentanol, hexanol, ethylene glycol, propylene glycol, dipropylene glycol, glycerin erythritol, xylitol, mannitol, diethylene glycol monoethyl ether, and mixtures thereof. In embodiments of the present disclosure, water soluble hydrocolloid polymers, for example, sodium alginate, xanthan gum, waxy maize starch, pectins, agar, carrageenan, various starches, furcelleran, larch gum, guar gum, locust bean gum, among others, are mixed into the polar phase.

An emulsifier (or “emulsifying agent”) can be added to the compositions. The term “emulsifier,” as used herein, refers to a compound that has a hydrophilic moiety and a hydrophobic moiety and can be used to form a stable mixture of two or more immiscible phases, such as a hydrophobic liquid and a hydrophilic liquid. In certain embodiments, these compounds are added to lower the interfacial tension between the aqueous phase and the oil phase of the formulation. The formulations of embodiments described herein can utilize less surfactant than traditional formulas.

Emulsifiers can be derived from natural sources, or they can be chemically synthesized. Exemplary emulsifiers include, for example, lecithins (such as sunflower lecithin or soy lecithin), mono- and di-glycerides of fatty acids, sodium stearoyl lactylate (SSL), acacia gum, beta-cyclodextrin, xanthan gum, guar gum, cyclodextrin, carrageen, gum arabic, purity gum ultra, polysorbate 80, Q-natural, xanthan gum, Ticamulsion, gellan gum, and combinations thereof.

The surfactants and/or emulsifiers can be added to the nonpolar phase, the polar phase, or they can be added later when the nonpolar and polar phase are in the mixing stage in order to form an emulsion. The choice of where and when to add these additional compounds can depend on the hydrophobicity of the compounds themselves.

Sugar esters can also be used as surfactants (Szuts et al., (2007), “Study of thermal behaviour of sugar esters” International Jour. Pharm., 336:199-207) due to their pleasant taste and aroma profile, low toxicity, and high biodegradability compared to petrochemical-based surfactants such as polysorbate 80 (Sadtler et al., (2004), “Shear-induced phase transitions in sucrose ester surfactant”, Jour Colloid Interface Sci. 270:270-275).

Suitable surfactants that also act as emulsifiers include, for example sucrose monoesters of palmitic acid, lauric acid, and stearic acid. Sucrose monoesters are excellent emulsifiers and can be used at very low amounts to emulsify high oil loads. Sucrose esters of palmitic acid, lauric acid, and stearic acid mainly consist of monoesters of palmitic, lauric acid, and stearic acid respectively with smaller amounts of their diesters. They have a high hydrophobic-lipophilic balance (HLB) value that gives them greater emulsification properties in systems that require higher HLBs. The sucrose monoesters of palmitic acid, lauric acid, or stearic acid have excellent emulsifying properties and can be used at low levels to successfully emulsify the ingredients in the formulation.

Other suitable emulsifiers include lecithins. Lecithins are a heterogeneous group of fatty substances found in plant and animal tissues and they may be used in the preparation of foods, cosmetics, and medications, among other products. Lecithins may be derived from plant or animal sources such as eggs, soy, sunflower seeds, canola, cottonseed, rapeseed, or other plant or animal fats. Lecithins may contain naturally occurring heterogenous mixtures of one or more glycerophospholipids including phosphatidylcholine, phosphatidylethanolamine, phosphatidylinositol, phosphatidylserine, and phosphatidic acid. Commercially, lecithins are useful for emulsification, homogenization, and smoothing food textures. Lecithins can be completely metabolized by humans and are thus nontoxic and well tolerated when added to food and beverages.

Soybean lecithin is a blend of phospholipids that naturally occurs in soybean. Soybean lecithin is amphiphilic, having an affinity for both oil and water. Accordingly, soybean lecithin can be added in either oil or water phase during processing. In an embodiment, soybean lecithin is added to the oil phase.

Soybean lecithin is rich in three main phospholipids. Phosphatidylcholine is an amphiphilic molecule and is the primary emulsifier in lecithin. Phosphatidylethanolamine may mediate attaining smaller particle sizes since it has a smaller chain length than the other phospholipids in lecithins. Phosphatidic acid has a negative charge on its surface which helps to increase the electrostatic repulsion between oil droplets and therefore prevents agglomeration and coalescence of the droplets. Phosphatidic acid content in lecithin is useful for obtaining stable emulsions.

In an embodiment soybean lecithin advantageously contains all three of the above phospholipids. In an embodiment, soybean lecithin contains at least 70% phosphatidylcholine. Soybean lecithin also helps to emulsify high oil loads.

Sunflower lecithins may be used as an alternative to, or in conjunction with, soybean lecithins and may be advantageous for avoiding allergies to soy for some consumers. Additional advantages include its high phosphatidylcholine content, lower linolenic acid content which may promote long term stability, and natural non-GMO sources for sunflower lecithin.

Egg lecithin is an additional source for use of lecithin as an emulsifier. Egg lecithin also contains high levels of phosphatidylcholine as well as phosphatidylethanolamine and sphingomyelin. Egg lecithin may be preferred to soybean lecithin for avoiding allergies to soy for some consumers.

Thickening agents (also called texture modifiers or bulking agents) are a type of stabilizer that can be used in addition to emulsifiers. Thickening agents can improve emulsion stability by retarding droplet movement, but they may also be used to provide desirable textural characteristics, such as “creaminess,” “richness,” “thickness,” or gel strength. Most texture modifiers used in the food industry are biopolymers, such as proteins (e.g., egg, milk, vegetable proteins) or polysaccharides (e.g., starch, pectin, alginate, carrageenan, xanthan, guar gum). Certain thickening agents, such as pectin and xanthan gum, are able to stabilize emulsions and provide an additional layer of protection against phase separation. Other suitable bulking agents can also be used.

In some implementations, the oral thin film formulations disclosed herein may comprise one or more hydrocolloid film-forming agents. Hydrocolloids are a heterogeneous group of long chain polymers characterizes by their property of forming viscous dispersions and/or gels when dispersed in water. Hydrocolloid polymers may include, for example, polysaccharides and proteins. In some implementations, hydrocolloid film-forming agents included in the oral thin film formulations disclosed herein may include extracts from land plants and marine algae, e.g., pectins, agar, alginate, carrageenan, various starches, cellulose, furcelleran, larch gum, among others. Such plant-derived hydrocolloids may function as scaffolding substances that form a matrix when dispersed in an aqueous solution.

TABLE 2
(from Karaman et al.)

| Sample | Specific gravity | Moisture (%) | pH | WHC | OHC | a_{w} | Ash (%) |
|---|---|---|---|---|---|---|---|
| Alginate | 1.019^{a} | 5.260^{a} | 9.570^{a} | 40.000^{a} | 0.850^{a} | 0.329^{a} | 20.318^{a} |
| CMC gum | 0.431^{b} | 12.767^{b} | 6.637^{b} | 10.000^{b} | 1.400^{b} | 0.357^{b} | 20.781^{b} |
| Guar gum | 0.746^{c} | 10.250^{c} | 5.860^{c} | 4.800^{c} | 1.067^{c} | 0.372^{c} | 0.922^{c} |
| Tara gum | 0.800^{d} | 10.130^{c} | 5.923^{d} | 17.133^{d} | 1.400^{b} | 0.323^{a} | 1.030^{c} |
| Locust bean gum | 0.992^{e} | 10.660^{c} | 5.470^{e} | 5.600^{e} | 0.600^{d} | 0.393^{d} | 1.178^{c} |
| Carrageenan | 0.848^{f} | 9.330^{d} | 9.830^{f} | 11.733^{f} | 1.267^{b} | 0.287^{e} | 20.051^{d} |
| Xanthan gum | 0.713^{g} | 10.740^{c} | 7.900^{g} | 19.200^{g} | 1.567^{e} | 0.342^{f} | 11.796^{e} |

Hydrocolloids may be characterized by their water holding capacity (WHC), the quantification of the water bound by a film-forming agent and expressed as grams of water per 100 grams hydrocolloid. WHC may be determined, for example, by the methods of Omojola et al. (see Omojola et al., (2010), “Isolation and Physico-chemical characterization of cola starch”, African Journal of Food Agriculture Nutrition and Development, 10:7, 2884-2900.), wherein the dry hydrocolloid sample is dispersed in a pre-weighed centrifuge tube containing water. The tube is agitated in a vortex mixer for two minutes. The supernatant is then discarded and the weight of the tube and the hydrated sample is then record. The weight is calculated and expressed as the weight of water bound by 100 g dry hydrocolloid sample. The weight of water=weight of the tube+hydrated sample−supernatant. Among various hydrocolloids, the WHC of several hydrocolloids are, for example, as follows: for alginate, 40.0 g water/110 g (see Karaman et al., (2014), “Rheological and some Physiochemical Properties of Selected Hydrocolloids and their Interactions with Guar Gum: Characterization using Principal Component Analysis and Viscous Synergism Index”, International Journal of Food Properties, 72:1655-1667); for xanthan gum, 19.0 g water/100 g (Karaman et al.); for maize starch, 93.0 g water/100 g (see Kolawole et al., (2013), “Comparison of the Physicochemical Properties of Starch from Ginger (Zingiber officinale) and Maize (Zea mays).”, International Journal of Science and Research, 2:11, 71-75).

In some implementations, it may be desirable to select film-forming agents with relatively low water-holding capacities. While not wishing to be bound by any particular theory, it is believed that film-forming agents with high WHCs may absorb and consume the available water in a formulation, thereby leaving less water available to solubilize additional water-soluble active ingredients. By choosing film-forming agents with lower WHCs, more water is left available in the formulation to solubilize additional water-soluble active ingredients. In certain implementations, film-forming agents are chosen that have WHCs of less than 100.0 g water/100 g film-forming agent. In preferred implementations, film-forming agents are chosen that have WHCs of less than 50.0 g water/100 g film-forming agent. In certain implementations film-forming agents are chosen that have WHCs of 10.0-20.0 g water/100 g; 20.0-30.0 g water/100 g; 30.0-40.0 g water/100 g; 40.0-50.0 g water/100 g; or value therebetween. In preferred implementations, one or more film-forming agents having a certain WHC may be used in combination with one or more additional film-forming agents having a different WHC, e.g., one or more film-forming agents having a WHC of less than about 50.0 g water/100 g film-forming agent may be used in combination with one or more film-forming agents having a WHC of about 50.0-60.0 g water/100 g; 60.0-70.0 g water/100 g; 70.0-80.0 g water/100 g; 80.0-90.0 g water/100 g; 90.0-100.0 g water/100 g; or values therebetween.

Hydrocolloids may also be characterized by their oil holding capacity (OHC), the quantification of the oil bound by a film-forming agent and expressed as grams of oil per 100 grams hydrocolloid. Among various hydrocolloids, the OHC of several hydrocolloids are, for example, as follows: for alginate, 0.85 g oil/100 g (Karaman et al.); for xanthan gum, 1.567 g oil/100 g (Karaman et al.). Notably, xanthan gum has the highest OHC of the hydrocolloids described by Karaman et al.

In some implementations, it may be desirable to select film-forming agents with relatively high oil-holding capacities. While not wishing to be bound by any particular theory, it is believed that film-forming agents with high OHCs may promote the absorption and retention of oil in the matrix of a formulation, allowing oil-based plant extracts or oil-based active ingredients to be retained in the final dried OTF strip. Applicant has observed that for formulations utilizing film-forming agents with lower OHCs, oil may leave the matrix during drying, resulting in an unsatisfactory product with pooled oil on the dried surface of the strip. By choosing film-forming agents with higher OHCs, oil is retained in the matrix during drying, resulting in an adequately dried, stable, flexible OTF strip. In certain implementations, film-forming agents are chosen that have OHCs of greater than about 0.60 g oil/100 g; 0.70 g oil/100 g; 0.80 g oil/100 g; 0.90 g oil/100 g; 1.0 g oil/100 g; 1.1 g oil/100 g; 1.2 g oil/100 g; 1.3 g oil/100 g; 1.4 g oil/100 g; or 1.5 g oil/100 g film-forming agent. In certain implementations, film-forming agents are chosen that have OHCs of greater than 0.6-0.7 g oil/100 g film-forming agent; 0.7-0.8 g oil/100 g film-forming agent; 0.8-0.9 g oil/100 g film-forming agent; 0.9-1.0 g oil/100 g film-forming agent; 1.0-1.1 g oil/100 g film-forming agent; 1.1-1.2 g oil/100 g film-forming agent; 1.2-1.3 g oil/100 g film-forming agent; 1.3-1.4 g oil/100 g film-forming agent; 1.5-1.6 g oil/100 g film-forming agent; 1.6-1.7 g oil/100 g film-forming agent; 1.7-1.8 g oil/100 g film-forming agent; 1.8-1.9 g oil/100 g film-forming agent; or 1.9-2.0 g oil/100 g film-forming agent, or values therebetween. In certain implementations, film-forming agents are chosen that have OHCs of greater than 2.0 g oil/100 g film-forming agent.

Hydrocolloids may also be characterized by their viscosity, the quantification of the resistance to deformation of a fluid at a given rate and expressed as centipoise (cp), the amount of force necessary to move a layer of liquid in relation to another liquid. A centipoise is equivalent to one millipascal-second in SI units. Viscosity of a hydrocolloid may be measured, for example, in the context of a given percentage aqueous solution of the hydrocolloid at a given temperature. Among various hydrocolloids, the viscosity of a 1% aqueous solution at 30° C. are, for example, as follows: for pullulan, 2 cp; for sodium alginate, 200-700 cp; for xanthan gum, 2,000-3,000 cp (Tsujisaka et al.).

While not wishing to be bound by any particular theory, it is believed that the viscosity of film-forming agents is related to their WHC and OHC, and these variables in combination affect the properties of the final dried OTF strip and the number and abundance of water-soluble and oil-soluble active ingredients that may be retained in the final dried OTF strip while maintaining a stable matrix. In certain implementations, one or more film-forming agents are chosen that have viscosities of less than 1000 cp. In certain implementations, film-forming agents are chosen that have viscosities of 100-200 cp; 200-300 cp; 300-400 cp; 400-500 cp; 500-600 cp; 600-700 cp; 700-800 cp; or 800-900 cp. In other implementations, one or more film-forming agents are chosen that have viscosities of 1000-5000 cp. In certain implementations, one or more film-forming agents are chosen that have viscosities of 1000-2000 cp; 2000-3000 cp; 3000-4000 cp; or 4000-5000 cp. In preferred implementations, one or more film-forming agents of a certain viscosity may be used in combination with one or more additional film-forming agents of a different viscosity, e.g., one or more film-forming agents with a viscosity of less than 1000 cp may be used in combination with one or more film-forming agents with a viscosity of 1000-2000 cp; 2000-3000 cp; 3000-4000 cp; or 4000-5000 cp.

In a preferred implementation, alginate is utilized as a film-forming agent in the oral thin film formulations disclosed herein. For example, sodium alginate is soluble in cold water and may form gels when mixed with water without a need for additional heating. Gels comprising water and alginate are thus heat stable. This property of alginate is advantageous for incorporating additional active ingredients which may be susceptible to degradation when exposed to higher gel activation temperatures, as are required for some other hydrocolloids, e.g., pullulan.

Starch is a polymeric sugar moiety used for energy storage in plants and can be found, for example, in wheat, potatoes, maize, rice, and cassava. Once processed from the plant source, starch is a white, tasteless and odorless powder. Starch can vary depending on the plant source and processing method. In an embodiment of the disclosure, the bulking agent is a maize or rice starch.

In some implementations, modified waxy maize starch may be used as a film-forming agent in the microemulsion formulations. Modified waxy maize starch can be modified, for example, to form a starch alkenyl succinate ester. These compounds are usually prepared commercially by the base catalyzed reaction of alkenyl succinic anhydrides with granular starch in aqueous suspension, and can result in stronger emulsification properties (Shogren et al, (2000), “Distribution of Octenyl Succinate Groups in Octenyl Succinic Anhydride Modified Waxy Maize Starch”, Starch/Starke 52:196-204). Modified waxy maize starch may comprise about 1%-2%, 2%-3%, 3%-4%, or about 4%-5% (w/w) of the final dried OTF strip.

The bulking agent can also be a starch derived from cassava. Cassava (Manihot esculenta) is a starchy root vegetable that is native to South America and is widely used as a staple food in many countries of the world. The properties of starch from cassava have been described, for example, in Oladunmoye et al., (2014), “Chemical and functional properties of cassava starch, durum wheat semolina flour, and their blends”, Food Sci Nutr. 2:132-138.

Carrageenans are a family of linear sulfated polysaccharides that are extracted from red edible seaweeds. They may be used in food applications for the gelling, thickening, and stabilizing properties. An advantage of carrageenans are that they are plant-derived and may be used as a vegan alternative to gelatin in some implementations, which is desirable for some consumers and may have market advantages.

In some implementations, xanthan gum may be used in the microemulsion formulations as a film-forming agent. Xanthan gum is a polysaccharide that may be used in food applications as a thickening agent and stabilizer to prevent the separation of component ingredients in a mixture. Xanthan gum may be produced from simple sugars using a fermentation process. Xanthan gum is particularly useful for increasing the viscosity of a liquid to which it is added. Xanthan gum may stabilize and thicken an emulsion to which it is added, with increasing concentration of xanthan gum correlating to a thicker, more stable matrix for the final dried OTF strip. Xanthan gum may comprise about 0.1%-0.2%, 0.2%-0.3%, 0.3%-0.4%, 0.4%-0.5%, 0.5%-0.6%, 0.6%-0.7%, 0.7%-0.8%, 0.8%-0.9%, or about 0.9%-1.0% (w/w) of the final dried OTF strip.

Pectin is a structural acidic heteropolysaccharide derived from the cell walls of plants. Commercially, pectin is typically extracted from citrus fruits and may be used in food and beverage applications as a gelling agent, thickening agent, and stabilizer.

Guar gum is a galactomannan polysaccharide extracted from guar beans that may be used as a thickening agent and stabilizer in food and beverage applications. An advantage of guar gum is that it shows a clear low shear plateau on the flow curve and is strongly shear-thinning, meaning its viscosity decreased under shear strain. Guar gum may exhibit viscosity synergy with xanthan gum, increasing viscosity of the liquid to which it is added greater than expected in the absence of an interaction between the two components.

In some implementations, alginate may be used in the microemulsion formulations as a film-forming agent. In a preferred implementation, sodium alginate may be used in the microemulsion formulations as a film-forming agent. Alginate is a natural acidic polysaccharide derived from brown seaweeds that may be used as a thickening agent, gelling agent, and stabilizer. An advantage of alginate is its unique gelling abilities at low temperatures. Alginate may be used in microparticle formation, whereby emulsion droplets are used as a template for the formulation of alginate nanospheres, with various applications in food, beverage, and pharmaceutical formulations. Sodium alginate may comprise about 10%-11%, 11%-12%, 12%-13%, 13%-14%, 14%-15%, 15%-16%, 16%-17%, 17%-18%, 18%-19%, or about 19%-20% (w/w) of the final dried OTF strip.

Plasticizers can also be used in the sublingual formulations. Suitable plasticizers, such as sorbitol, glycerin, and polyethylene glycol can be used.

Glycerin is not only useful as a plasticizer, but can also be useful as a humectant, allowing the stored thin strips to maintain a desired moisture level. This prevents cracking, maintains pliability, prevents discoloration, and otherwise prevents degradation of the prepared OTF strip. In an implementation, glycerin may also function as a film-forming agent in the microemulsion formulations. Glycerin may comprise about 5%-6%, 6%-7%, 7%-8%, 8%-9%, or about 9%-10% (w/w) of the final dried OTF strip.

In some implementations, it may be desirable to decrease the quantity, as a percentage of the formulation by weight, of the film-forming agents used so as to allow an increased quantity and abundance, as a percentage of the formulation by weight, of additional water-soluble or oil-soluble active ingredients, or both. For example, for a formulation using sodium alginate as a film-forming agent, sodium alginate may be present in the formulation at about 30-40% in (w/w) of the final dried OTF strip to produce a stable and flexible matrix. However, in order to incorporate additional water-soluble or oil-soluble active ingredients, the percentage of sodium alginate may be reduced in the formulation to about 15-20% (w/w) of the final dried OTF strip. Due to the mechanisms discussed above, e.g. its WHC, OHC, and viscosity of 200-700 cp, however, a formulation comprising 15-20% (w/w) sodium alginate after drying would not yield a stable matrix suitable for an orally consumable oral thin strip. In such an implementation, xanthan gum may be included as an additional film-forming agent in combination with sodium alginate, comprising 0.4-0.5% (w/w) of the final dried OTF strip. Due to the mechanisms discussed above, e.g. its WHC, OHC, and viscosity of 2,000-3,000 cp, this amount of xanthan gum is, in combination with 15-20% (w/w) sodium alginate, is sufficient to form a stable, robust, and flexible matrix in the final dried OTF strip while decreasing the relative amount of total film-forming agent and thus allowing for the incorporation of additional water-soluble or oil-soluble active ingredients.

In some implementations, waxy maize starch may be utilized in the formulation as an additional film-forming agent. Due to its relative high oil-holding capacity (OHC), waxy maize starch may be included in the formulation to enhance the oil retention qualities of the formulation and allow for an increased abundance of oil-soluble active ingredients retained within the matrix of the formulation without substantially affecting the viscosity of the formulation. In an implementation, waxy maize starch may be present in the formulation at about 1-5% (w/w) of the final dried OTF strip.

In a preferred implementation, modified waxy maize starch, sodium alginate, xanthan gum, and glycerin may be used in combination as film-forming agents in the microemulsion formulations. While not wishing to be bound by any particular theory, due to the mechanisms discussed above, e.g. the WHC, OHC, and viscosity of these film-forming agents, when utilized in combination in the amounts disclosed herein, e.g., 15-20% (w/w) sodium alginate, 0.4-0.5% (w/w) xanthan gum, 1-5% (w/w) waxy maize starch, and 5-10% (w/w) glycerin in the final dried OTF strip, applicant has been able to maximize the number and abundance of additional water-soluble or oil-soluble active ingredients in the formulation while producing a stable matrix underlying an orally dissolvable thin strip.

In an implementation, the total combination of one or more film-forming agents may comprise 5%, 10%, 15%, 20%, 25%, 30%, or 35% of the dried OTF strip (w/w). In a preferred implementation, the total combination of one or more film-forming agents comprises less than 35% of the dried OTF strip (w/w). In a further preferred implementation, the total combination of one or more film-forming agents comprises less than 30% of the dried OTF strip (w/w).

While not wishing to be bound by a particular theory, Applicant has discovered that the preferred combination and usage rate of the one or more film-forming agents, i.e., in some implementations less than 35% of the dried OTF strip (w/w), allows for the formation of a robust matrix for the dried OTF strip while simultaneously leaving enough free aqueous phase and oil phase to solubilize a higher number and quantity of active ingredients than in previously disclosed formulations.

Other ingredients that can be used as a plasticizer include, for example, vitamin E succinate (VES), acetyltributyl citrate (ATBC), triethyl citrate (TEC), triacetin and polyethylene glycol 8000 (PEG 8000) (Thumma et al., (2008), “Influence of Plasticizers on the Stability and Release of a Prodrug of Δ9-Tetrahydrocannabinol Incorporated in Poly (Ethylene Oxide) Matrices”, (Eur J Pharm Biopharm., 70:605-614).

Due to the low water activity of the sublingual film, preservatives do not necessarily need to be added to the formulations. In a preferred embodiment, the OTF formulation is preservative-free. This is beneficial as many consumers prefer preservative-free products. The oral strip products described herein are generally stable for at least about 6 months, 1 year, 1.5 years, or 2 years. In an embodiment of the disclosure, the oral strip product is stable for at least about 1 year, without the need for preservatives, due, at least in part, to the low water activity of the formulation. Preservatives can be added if desired, however, and will likely increase the stability of the product. Suitable food grade preservatives include, for example, sodium benzoate, potassium sorbate, acetic acid, citrus extract, citric acid, and mixtures thereof.

Flavoring agents can be added to the formulation. The flavoring agents can be natural or artificially derived. For example, herbal or spice flavors such as mint, sage, onion, licorice, garlic, vanilla extract, and rum extract can be added. Fruit or flower flavors such as strawberry, cherry, citrus, apple, berry, lavender black currant, orange, mint, rose, among others can be added. Other flavors can also be added to the formulation, as desired.

Sweetening agents can be added to the formulation and can be natural or artificial. Exemplary sweetening agents include, for example, sugar, fructose, glucose, sucrose, sorbitol, xylitol, mannitol, honey, aspartame, stevia (and derivatives), sucralose, saccharin, cyclamate, corn syrup, fruit-based sugar syrup concentrates, and mixtures thereof. In some embodiments, no sugar or sweetener is added.

Coloring agents can be added to the formulation. The coloring agents can be natural coloring agents, or they can be synthetic coloring agents. Suitable naturally derived colorants, such as anthocyanins, carotenoids, betalains, and chlorophylls, are described, for example, in Sigurdson et al, (2017), Natural Colorants: Food Colorants from Natural Sources”, Annu Rev Food Sci Technol., 8:261-280.

In an embodiment of the disclosure, active ingredients in addition to the cannabinoid(s) can also added to the formulation to create an energy boost upon administration of the thin oral strip. Exemplary ingredients include, for example, ingredients having naturally high amounts of caffeine, such as natural green tea extract and Yerba mate extract. Addition of caffeine from other sources can also be used. A mixture of energy enhancing agents can be used. The amount of caffeine per oral strip can be adjusted as desired. In an embodiment, each oral strip has a caffeine loading of from about 0.01, 0.05, 0.1, 0.5, 1.0, 1.5, 2.0, to about 2.5 mg per serving. In another embodiment, each oral strip has a caffeine loading of from about 1.5, 2, 4, 6, 8, 10, 12, 14, 16, 18, 20, 22, or 25 or more mg per serving. Other energy-enhancing agents can be added, such as, for example, ginseng, guarana, taurine, gluconolactone, among other energy-enhancing agents.

The energy-enhancing agent caffeine can be added to the formulation. It can be added as purified powder form. Caffeine can also be added from a plant extract, or as a semi-purified mixture (such as, for example, a powder or extract made from black tea, green tea, or other caffeine-rich plants). Caffeine can be found, for example, in cocoa beans, kola nuts, tea leaves, coffee beans, and yerba mate. Caffeine is also present in the berries of the Amazonian stimulant plants guarana (Paullinia cupana) and guayusa (Ilex guayusa).

Green tea or black tea (both typically derived from Camellia sinensis) are widely available and are a good source of caffeine and other bioactive compounds (e.g., polyphenols and carotenoids). Alternatively, unroasted green coffee beans are an additional source of caffeine which may be used in formulations disclosed herein.

Caffeine may recrystallize out of solution in nanoemulsion formulations, which presents a problem for the texture and solubility of oral thin strips. Recrystallization may also have detrimental effects on the mechanical and mucoadhesive properties of the oral thin strip formulations, on active ingredient content uniformity throughout the strip, and on active ingredient release and associated kinetics as the thin strip dissolves and active ingredients cross the sublingual epithelium. The methods disclosed herein minimize the recrystallization of caffeine to maintain the flexibility of rapid dissolution of the oral thin strips for a consistent and effective user experience.

An additional approach to minimizing caffeine recrystallization is to modulate the water content of the oral thin strip formulation. Alternative matrix components, i.e. substituting modified waxy maize starch for sodium alginate, may require less water for dissolution and promote the solubility of caffeine and mitigate risk of caffeine recrystallization.

Guarana (Paullinia cupana) is rich in several bioactive compounds such as methylxanthines (including caffeine, theobromine and theophylline) tannins, saponins, catechins, epicatechins and proanthocyanins. Guarana is associated with protection against hypertension, obesity and metabolic syndrome in elderly healthy volunteers (Lima et al., (2017), “Modulatory Effects of Guarana (Paullinia cupana) on Adipogenesis”, Nutrients, 9:635).

The Amazonian plant Guayusa is a good source of caffeine and other beneficial bioactive compounds (Gan et al. (2018), “Health Benefits of Bioactive Compounds from the Genus Ilex, a Source of Traditional Caffeinated Beverages” Nutrients, 10:1682).

An extract or powder from Yerba mate (Ilex paraguariensis) can be added to the formulation. Yerba mate is widely used in South America, either as a tea, other beverage, or as a nutritional supplement. In addition to the presence of energy enhancing caffeine, it can also be useful as a hypocholesterolemic agent, hepatoprotective agent, central nervous system stimulant, and a diuretic. It is also thought to benefit the cardiovascular system, and may be useful in battling obesity (see Heck et al., (2007), “Yerba Mate Tea (Ilex paraguariensis): a comprehensive review on chemistry, health implications, and technological considerations”, Journal of Food Science, 72(9):R138-151).

The energy-boosting formulation can also have other ingredients such as vitamins. In an embodiment, the formulation also contains multivitamins or certain soluble vitamins, and/or soluble B vitamins.

In another embodiment of the present disclosure, active ingredients are also added to the formulation to induce sleep. In an embodiment, the sleep enhancing strip is formulated with THC, CBD, and at least one sleep enhancing agent. For example, in an embodiment, each serving of the formulation contains about 5 mg THC, 5 mg CBD, and one or more sleep enhancing agents. Exemplary sleep-enhancing agents include, for example, GRAS sleep aids, such as lavender, valerian, melatonin, L-tryptophan, and L-theonine.

Lavender, a common name for several species of plants belonging to the genus Lavendula in the mint family, has been found by some to have sleep-enhancing or calming effects. Thus, an extract of lavender can be useful as a component of the oral thin strip.

Valerian is an herb that is native to Asia and Europe, and has long been used as a medicinal plant, useful as a sleep aid, and also for treatment of symptoms such as anxiety, depression, and menopause. For a review and meta-analysis, see Fernandez et al, (2010), “Effectiveness of Valerian on insomnia: a meta-analysis of randomized placebo-controlled trials”, Sleep Medicine, 11:505-511). A powder or extract of the plant can be added to the sublingual strip formulation.

Melatonin is a hormone that is naturally produced in the human body, being mainly released by the pineal gland. Natural melatonin synthesis and secretion in humans is enhanced by darkness and inhibited by light. Its secretion starts soon after sundown, reaches a peak in the middle of the night. As a supplement, intake of a usual dose of about 1 to 5 mg results in melatonin concentrations 10 to 100 times higher than the physiological nocturnal peak, at about 1 hour after intake (Tordjman et al (2017), “Melatonin: Pharmacology, Functions and Therapeutic Benefits”, Current Neuropharmacology, 15:434-443). Melatonin is commercially available over the counter as a sleep supplement, and can be used for inducing sleep, especially for the short-term treatment of trouble sleeping due to a change in circadian rhythm, such as from jet lag or shift work.

The amino acid L-theanine has also been found to have sleep enhancing properties. A recent research publication demonstrated the sleep-promoting effects of dietary theanine in Drosophila, showing that the amino acid caused a markedly increased daily amount of sleep and also decreased the latency to sleep onset in a dose-dependent manner (Ki and Lim (2019), “Sleep-promoting effects of theanine link amino acid metabolism in Drosophila neuron to GABAergic control of sleep drive”, Elife, 8: e40593).

The above-described sleep-enhancing compounds, either alone or in a mixture with other sleep enhancing compounds, can be particularly useful when administered in an oral thin strip form. The sleep-enhancing agents can be derived from plant sources, microbial sources, or other sources such as synthetic sources. In an embodiment, the sleep-inducing agent is a natural ingredient, such as an extract of a plant. In another embodiment, the sleep-enhancing agent is isolated and purified. Other sleep-enhancing agents can be added, such as, for example, chamomile extract, ashwagandha, doxylamine succinate, diphenylhydramine, among other sleep-enhancing agents.

In a further embodiment of the disclosure, the thin strip is formulated so that a relatively large amount of THC can be successfully and efficiently administered using an oral thin strip. Some benefits of this formulation variant include fast uptake by the epithelial layers of the tongue, improved predictability of effect, and more rapid onset than other oral THC products. Rapid dissolution of the oral thin strip formulations described herein promote a high concentration of dissolved formulation at the epithelial layer of the sublingual tissue, thereby generating a substantial concentration gradient across the epithelial layer and rapid transcellular transport of active ingredients into the blood stream. The formulation allows for modulation of the amount of THC to be administered per strip (such as, for example, low dose, medium dose, high dose), as desired for specific target needs. The high THC strip can be formulated to have about 1 mg, 2 mg, 3 mg, 4 mg, 5, mg 6 mg, 7 mg, 8, mg, 9 mg, 10 mg, 11 mg, 12 mg, or more per strip (1 dose).

In some implementations, the microemulsion formulations and the final dried OTF strips may contain 1, 2, 3, 4, 5, 6, 7, 8, 9, 10 or more active ingredients. In an implementation, the total combination of the active ingredients may comprise 30%, 35%, 40%, 45%, 50%, or more of the dried OTF strip (w/w). In some implementations, the efficacy of each active ingredients is tunable without the need to alter the film forming agents. In another implementations, the efficacy of each active ingredients is tunable between 10% and 66% of the dried OTF strip (w/w) without the need to alter the film forming agents. In still another implementations, the efficacy of each active ingredients is tunable while the amounts of the film forming agents are altered less than about 1% of the dried OTF strip (w/w). In a preferred implementation, the total combination of the active ingredients comprises more than 40% of the dried OTF strip (w/w).

In some implementations, active ingredients may be either water soluble or oil soluble and are introduced to either the aqueous phase or the oil phase, respectively, before the mixing stages of the methods disclosed herein. Additional water soluble active ingredients that may be included in the microemulsion formulations include ginger, turmeric, blueberry extract, elderberry extract, grapefruit powder, rosehip powder, asparagus powder, ginseng, Rhodiola rosea, hibiscus powder, acai berry extract, guayusa extract, hawthorn berry powder, amla extract, vitamin C, L-Glutamine (L-Glutamine hydrochloride), choline, bacopa monnieri extract, Magnesium, L-pyroglutamic acid, DMAE (Dimethyl amino ethanol Bitartrate), Ashwagandha (Indian ginseng), Ginkgo Biloba, Artichoke extract (Cynara scolymus), L-Tyrosine, creatine, Glutathione, among others.

Additional oil soluble active ingredients that may be included in the microemulsion formulations include cinnamon oil, cardamom oil, ginger oil, basil oil, chamomile oil, clove oil, lavender oil, rosemary oil, chamomile oil, Ylang Ylang, bergamot oil, fennel, vitamin D, vitamin A, Vitamin E, cacao, omega-3 oil, bacopa monnieri extract, vinpocetine, Huperzine A, neem oil, lemon oil., among others.

While not wishing to be bound by a particular theory, Applicant has discovered that the microemulsion formulations and methods of preparation of dried OTF strips disclosed herein allow for the solubilization of a higher number and quantity (w/w) of active ingredients in the dried OTF strip than in previously disclosed formulations. In a preferred implementation, the ratio of active ingredients to film forming agent in the oral dissolvable thin film is at least 1.3:1 by weight. In some implementations, the ratio of active ingredients to film forming agent in the oral dissolvable thin film may be about 1.3:1, 1.4:1, 1.5:1 1.6:1, 1.7:1, 1.8:1, 1.9:1, or 2:1 by weight.

Antioxidants can also be added to the microemulsion formulations. Antioxidants can be used to prevent or slow degradation of the ingredients in the microemulsion formulations. Many antioxidants can be derived from edible plants, such as green tea extract, blueberry extract, goji extract, rosemary extract, ascorbic acid, carotenoids, green tea, black tea, fenugreek, grape seed, gotu kola, ginger, and gingko, among others. Additional antioxidants include, for example, vitamin E and TBHQ (Tertiary butylhydroquinone).

### Mixing

Once the nonpolar phase components are prepared and mixed, and the polar phase components are prepared and mixed, the nonpolar phase can be combined with the polar phase to form an emulsion. In an embodiment of the present disclosure, this is performed by high shear mixing, which can result in smaller emulsion particle sizes that are more stable.

In some implementations the mixed components of the oil phase can be gradually added to the mixed components of the aqueous phase to form an emulsion. In some embodiments, the oil phase, the aqueous phase, or both phases can be heated prior to or during the mixing process. The mixing can be performed at a desired speed or RPM.

In certain embodiments, the terms “high speed mixing” and “high shear mixing,” as used herein, refer to a step of mixing at an RPM (revolutions per minute) of greater than about 2,000 RPM. In an embodiment, the RPM during a high speed or high shear mixing step can be, for example, from about 2,000 RPM to 3,000 RPM, 3,000 RPM to 4,000 RPM, 4,000 RPM to 5,000 RPM, 5,000 RPM to 6,000 RPM, 6,000 RPM to 7,000 RPM, 7,000 RPM to 8,000 RPM, or more.

In general, miscible fluids, e.g. two or more polar solvents, or two or more hydrophobic oils, may be suitably combined by conventional mixing at lower RPMs, e.g. less than 1,000 RPMs, optionally with heating, while high-shear mixing is required to disperse a liquid phase or ingredient into a continuous phase with which it would normally be immiscible. Fluid undergoes shear when one area of fluid travels with a different velocity relative to an adjacent area of fluid. A high-shear mixer may be used to create emulsions, suspensions, homogenates, and to achieve particle size reduction and dispersion.

In some implementations high shear mixing of the combined oil phase and aqueous phase components is allowed to proceed for about 10, 11, 12, 13, 14, 15, 16, 17, 18, 19, 20, 21, 22, 23, 24, or 25 minutes.

An apparatus useful for high speed mixing can be a commercial mixing system, or a laboratory mixing system. Many types of suitable mixing systems are commercially available. In certain embodiments the Eurostar 20 Digital overhead stirrer (IKA Works, Inc., Wilmington, N.C.) may be used with various accessories including a dissolver stirrer, centrifugal stirrer, turbine stirrer, among others, to stir mixtures and introduce shear to mixtures.

In general, miscible fluids, e.g. two or more polar solvents, or two or more hydrophobic oils, may be suitably combined by conventional mixing at lower RPMs, optionally with heating, while high-shear mixing is required to disperse a liquid phase or ingredient into a continuous phase with which it would normally be immiscible. Fluid undergoes shear when one area of fluid travels with a different velocity relative to an adjacent area of fluid. A high-shear mixer may be used to create emulsions, suspensions, homogenates, and to achieve particle size reduction and dispersion.

The mixing can be performed, for example, at a range of from about 300 rpm, 400 rpm, 500 rpm, 600 rpm, 700 rpm, 800 rpm, 900 rpm, 1000 rpm, 2000 rpm, 3000 rpm, or 3,500 rpm or greater. The mixing can occur for a time range of from about 10′, 15′, 20′, 25′, 30′, 35′, 40′, 45′, 50′, 55′, to an hour or more. The mixing may be performed at approximately room temperature, i.e. at about 25° C.

In an embodiment, the average particle size in the emulsion after the mixing step is from about 0.1 μm to about 4 μm. In an embodiment, the average particle size is from about 0.1 μm, 0.2 μm, 0.3 μm, 0.4 μm, to about 0.5 μm, 0.6 μm, or about 0.8 μm. In an embodiment, the average particle size is from about 0.4 μm, 0.8 μm, 1.0 μm, 1.2 μm, 1.5 μm, 1.8 μm, 2.0 μm, to about 2.2 μm, 2.5 μm, 3.0 μm, 3.5 μm, or about 4.0 μm.

In an embodiment, the mixed liquid formulation may have an average particle size that is smaller than the average particle size of the lipophilic phase in the thin strip after drying. While not wishing to be bound by theory, this difference in average particle size between the mixed liquid formulation and the thin strip after drying may be due to some amount of coalescence of lipophilic microdroplets during the drying phase.

In an embodiment, an OTF composition is provided comprising at least one active ingredient and a film forming agent, having a percentage relative standard deviation of potency concentration of the at least one active ingredient in a batch of said oral dispersible film composition of less than 2.5%, preferably less than 1%, more preferably less than 0.5%.

The mixed formulation can then be poured onto a thin liquid layer to facilitate drying. The layer can be from about 200 μm to about 800 μm in thickness. For example, the layer can be from about 200 μm, 300 μm, 400 μm, 500 μm, 550 μm, 600 μm, 700 μm, and about 800 μm. In a preferred embodiment the layer is formed at thickness of about 550 μm. A film casting knife can be used, if desired, to facilitate the thickness of the thin liquid layer.

The liquid layer can then be dried to form a thin sheet having a low moisture content. In an embodiment, the layer is partially dehydrated using a commercial dehydrator system. Suitable commercial dehydrator systems can be obtained, for example, from companies such as Avantco, Turbo Air, Cabela, and Weston. The dehydration step can occur at room temperature, or with heating, such as from about 80° F., 85° F., 90° F., 95° F., 100° F., 105° F., 110° F., 115° F., and about 120° F. In a preferred embodiment, the dehydration step occurs at a temperature from about 80° F. to about 110° F. The formulation can be dried to a preferred moisture content, such as from about 3.5%, 4.0%, 4.5%, 5.0%, 5.5%, 6.0%, 6.5%, 7.0% water. The dehydration time can be adjusted as needed to result in the desired moisture content of the final product. For example, the dehydration step can be from about 15 minutes, 30 minutes, 45 minutes, 1 hour, 1.5 hours, 2.0 hours, 2.5 hours, 3.0 hours, 3.5 hours, 4.0 hours, 4.5 hours, and about 5.0 hours or more. This can depend on the temperature of the system, the humidity, the thickness of the formulation, the choice of ingredients in the formulation, and the final moisture content that is desired. In an embodiment, the dehydration step occurs at about 100° F. for about 2.5 hours.

The partially dehydrated thin layer can then be cut into strips that are suitably sized for sublingual administration, using a cutting device, such as a laser cutting instrument, knife, commercial shears, or other equipment. The length of the strip can be, for example, from about 0.5 inch, 0.75 inch, 1.0 inch, 1.2 inches, 1.5 inches, 1.75 inches, to about 2.0 inches. The width of the strip can be, for example, from about 0.3 inch, 0.5 inch, 0.75 inch, 1.0 inch, 1.25 inches, and about 1.5 inches. In a preferred embodiment, the strip is about 1.2 inches in length by about 0.75 inch in width.

The individual oral strips can then be packaged into airtight individual containers, which can be sold individually or in bulk packaging. In an embodiment, each strip is placed into a plastic pouch, and sealed with a heat sealer or other suitable device.

In an embodiment, the sublingual film formulation has a very low water activity. The low water activity of the sublingual film formulations described herein decreases the likelihood of contamination by microorganisms. Bacteria usually require a water activity of at least 0.91, and fungi at least 0.7. (Rockland et al, (1987), “Water Activity: Theory and Applications to Food” (2nd ed.). New York: Marcel Dekker). In an embodiment, the OTF strips may have a water activity 0.5 to 0.7. In a preferred embodiment the OTF strips may have a water activity of 0.21 to 0.66.

In an embodiment, the sublingual film formulations have an early onset of effect, in comparison to other oral cannabinoid products. While not being bound by theory, it is thought that this is likely to be due, in part, to the use of sublingual administration, where the actives are released immediately on dissolution and are carried through the epithelial layers of the tongue via a paracellular or transcellular route. This involves bypassing the first phase metabolism to ensure higher bioavailability and faster onset of the active ingredients.

The early onset of effect is desirable because without the early onset characteristic, the consumer may have an unpredictable experience, such as by possibly consuming more of a product than desired before the effect has initiated, assuming that it has a low potency. This can result in adverse side effects from overconsumption of active ingredients.

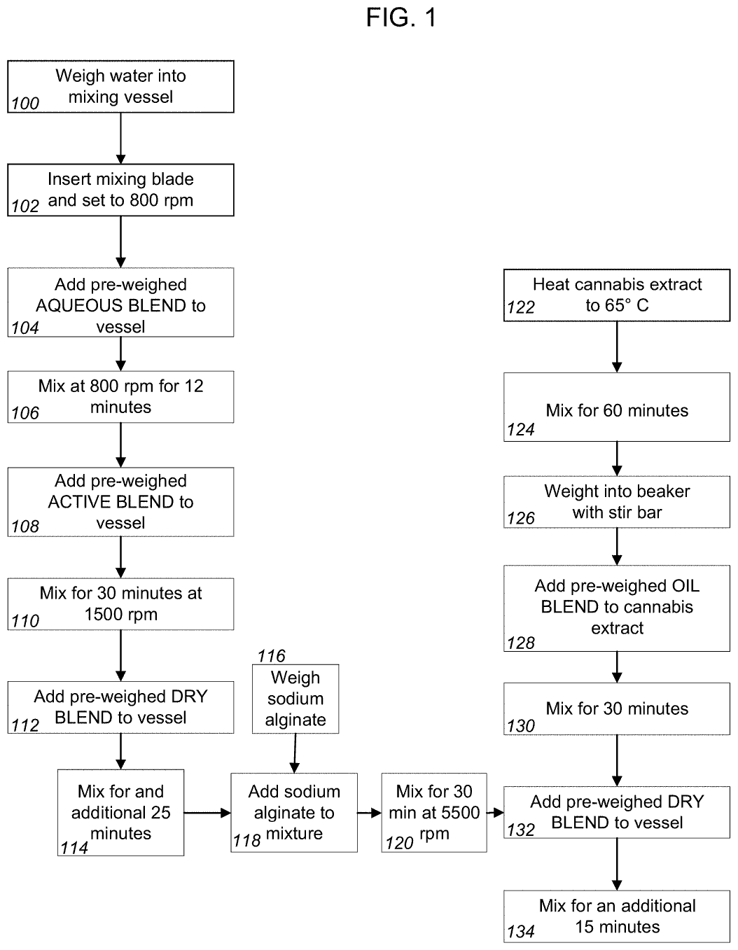

FIG. 1 is a process diagram depicting the mixing steps for various OTF formulations depicted herein. For various OTF formulations disclosed herein, the AQUEOUS BLEND 104, ACTIVE BLEND 108, DRY BLEND 112, and OIL BLEND 128 may each be comprised of different ingredients depending on the formulation.

Waters is weighed in mixing vessel 100 and mixing blade 102 is inserted into the mixing vessel and set to 800 rpm. Pre-weighed AQUEOUS BLEND 104 is added to the water as mixing continues at 800 rpm. Mixing 110 continues for an additional 12 minutes. Pre-weighed ACTIVE BLEND 108 is added to the mixing vessel. Mixing 110 speed is increased to 1500 rpm and continues for an additional 30 minutes. Pre-weighed DRY BLEND 112 is added to the mixing vessel and mixing 114 continues for an additional 25 minutes. Pre-weighed sodium alginate 116 is added to the mixture 118, and mixing speed 120 is increased to 5500 rpm and continues for an additional 30 minutes.

Meanwhile, Cannabis extract 122 is heated to 65° C. and mixed 124 for 60 minutes or until flowable. The Cannabis is weighed into a beaker 126 containing a magnetic stir bar and stirred at 200 rpm gently. Heated Cannabis extract 122 is added to pre-weighed oil blend 128 and stirring 130 continues for an additional 30 minutes.

The previously mixed water 100, AQUEOUS BLEND 104, ACTIVE BLEND 108, and DRY BLEND 112 are added to the mixed Cannabis extract 122 and OIL BLEND 128 and mixed 134 for an additional 15 minutes.

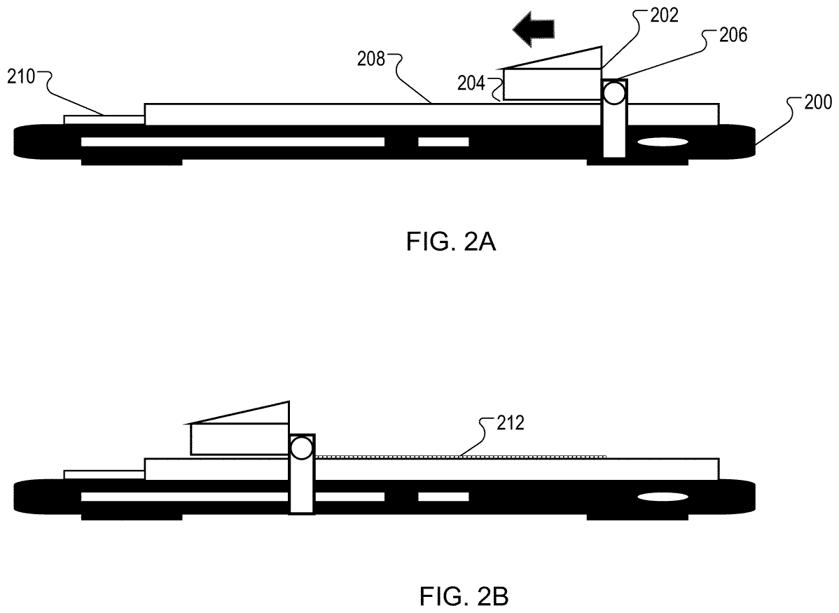

FIGS. 2A and 2B depict the film casting process for a preferred embodiment of the OTF formulations disclosed herein. In some implementations, the film casting process may be either manual or semi-automatic. In a preferred embodiment, the film casting process is semi-automatic, utilizing an electrically power automatic film casting knife apparatus. As depicted in FIG. 2A, automatic film casting knife apparatus 200 may be configured to cast the emulsion formulations disclosed herein into a thin film with a desired thickness, for subsequent dehydration and cutting into individual OTF strips. Blade applicator 202 is configured by the user to a predetermined film thickness 204, e.g. 550 μM. The liquid emulsified OTF formulation disclosed herein is loaded into blade applicator 202. Upon initiation of the casting process by the user, drive armature and applicator rod 206 are electrically drive in a right-to-left direction (indicated by the arrow), pushing bladed applicator 202 in a right-to-left direction at a user-defined speed, and spreading the liquid emulsified OTF formulation disclosed herein across substrate 208 at user-defined thickness 204, e.g. 550 μM. The thickness of the resulting film is defined by the gap between blade applicator 202 and substrate 208. When drive armature and applicator rod 206 reach the left side of automatic film casting knife apparatus 200, any excess liquid emulsified OTF formulation is captured by waste tray 210. As shown in FIG. 2B, the result of the film casting process is a thin film 212 comprising the emulsified OTF formulations disclosed herein spread across substrate 208 at a desired thickness 204, e.g. 550 μM. Substrate 208 and thin film 212 may be removed from automatic film casting knife apparatus 200 and subjected to additional processing steps, e.g. dehydration and laser-cutting.

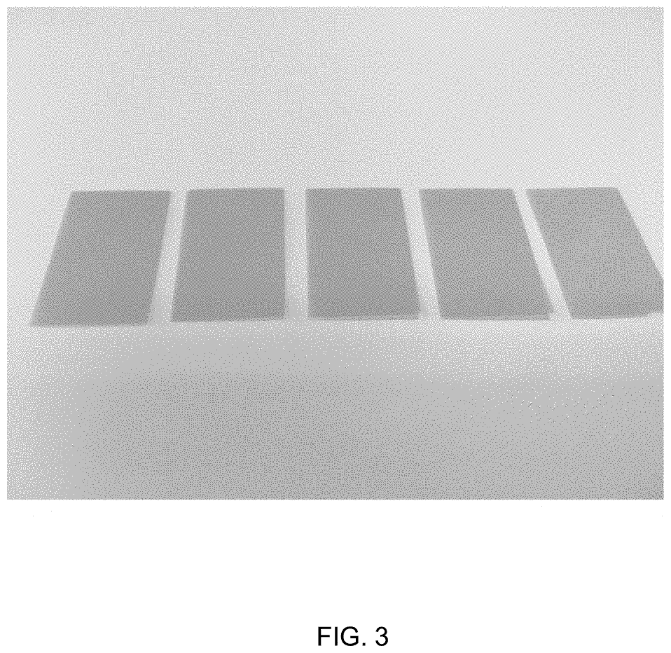

FIG. 3 is a photograph of a preferred embodiment of the semi-dehydrated OTF strips disclosed herein. In some implementations, the final semi-dehydrated OTF strips disclosed herein have a final thickness of about 550 μM. In some implementations, the final semi-dehydrated OTF strips have dimension of 1.2 inches by 0.75 inches. In some implementations, the final semi-dehydrated OTF strips have a residual moisture content of 5.5%±1%.

### EXAMPLES

### Comparative Example 1

An oral thin film composition and method of making the film was prepared according to published patent application US 2019/0269649. A mixture of 17.0 g glycerin and 19.0 PEG-1000, was prepared in a 100 mL glass beaker heated on a hot plate at 60° C. and stirred at 200 RPM substrate. To this mixture 40 mL of 60° C. water was added and stirring continued for 30-60 seconds until the components were evenly disbursed and homogenous. The mixture was transferred to a 1 L stainless steel cylinder.

500 mg of 100% purity crystallized caffeine was added to the mixture and stirred with a handheld high-shear mixer until the mixture was homogenous. To this mixture was added 5 g of 80% THC Cannabis extract and high-shear mixing was continued for an additional minute. While mixing continued, to the mixture was added 4.0 g A. M. Todd's Crystal White®, 6.0 g sucralose, 3.0 g acesufame potassium (ACE-K), and 3.0 g 50 μM microcrystalline cellulose (FMC's Endurance®). In a rapid fashion, over about 5-10 second, was added 24.6 g C. P. Kelco's Slow Set Genu® Pectin. High-shear mixing continued for an additional 3 minutes.

A film casting knife was affixed to a flat substrate, and the emulsion mixture was poured into the reservoir of the film casting knife and manually spread across the surface of the substrate. The film casting knife was drawn across the pour emulsion mixture to produce a uniform layer across the substrate and excess overflow emulsion mixture was collected in an additional vessel.

A commercial dehydrator was pre-heated to a temperature of 70° C. The substrate layered with the emulsion mixture was placed in the 70° C. pre-heated dehydrator and allowed to dry for 15 minutes. As the OTF formulation dried, opaque needle-like crystalline structures formed within the plane of the layer of emulsion mixture. An attempt was made to cut the resulting film into uniform individual 1.2″×0.75″ strips, but the precipitated crystalline structures interrupted the semi-dehydrated film and caused fracturing and brittleness of the resulting strips.

### Example 1

Quantities and proportions of ingredients for the following OTF formulation are described in Table 3 below.

Decarboxylated Cannabis extract was heated in an oven set at 60° C. until the fluid became flowable. Heated Cannabis extract was added to a pre-blended and pre-weighed oil phase mixture including MCTs, monoglycerides and diglycerides, terpenes, and polysorbate 80 according to the quantities described in Table 3. The combined oil phase mixtures and Cannabis extract were mixed at moderate speed until the Cannabis extract was completely dispersed into the oil phase mixture. As soon as the Cannabis extract was completely dispersed in the oil phase mixture, heating was ceased and mixing was allowed to continue at approximately 500 rpm as the mixture cooled to room temperature.

Concurrently the aqueous phase was prepared. Water was added to a mixing vessel and mixing speed was set to 500 rpm at room temperature. A blend of active ingredients including green tea extract, yerba mate extract, vitamin B complex were added to the aqueous mixture according to the quantities described in Table 3 and mixing speed was increased to 600 rpm. A pre-weighed blend of flavorants, plasticizers, and other aqueous phase components including vegetable glycerin, modified waxy maize starch, and neohesperidin dihydrochalone (NHDC) was added to the aqueous phase according to the quantities described in Table 3 and mixing continued at a speed of 600 rpm. Mixing of the aqueous phase including active ingredients continued at 600 rpm for 30 minutes. Mixing speed was reduced to 500 rpm and a mixture of sodium-alginate and xanthan gum was weighed according to the quantities described in Table 3. 50% of the sodium-alginate and xanthan gum mixture was added to the aqueous phase mixture and allowed to completely dissolve. Mixing speed was increased to 800 rpm and the remaining sodium-alginate and xanthan gum mixture was added and mixing continued at 500 rpm for 10 minutes. Mixing speed was increased to 1500 rpm to 2500 rpm and mixing of the aqueous phase continued for an additional 25 minutes. The blended oil phase was added to the aqueous phase while mixing continued at 1500-rpm 2500 rpm for an additional 25 minutes to produce a homogenous emulsified film mixture.

A film casting knife was affixed to a substrate and set to a thickness of 550 μM. The emulsion mixture was poured into the reservoir of the film casting knife and manually spread across the surface of the substrate. The film casting knife was drawn across the pour emulsion mixture to produce a uniform layer across the substrate and excess overflow emulsion mixture was collected in an additional vessel.

A commercial dehydrator was pre-heated to a temperature of 37.5° C. The substrate layered with the emulsion mixture was placed in the 37.5° C. pre-heated dehydrator and allowed to dry for 2 hours and 20 minutes, until reaching a final moisture concentration of about 5.5±1% water.

The dehydrated sheet was cut into individual 1.2″×0.75″ strips using conventional laser cutting equipment.

TABLE 3

| | % w/w in | Mg in a | % w/w in | Mg per |
|---|---|---|---|---|
| | mixed wet | 200 mg | dried final | dried final |
| Ingredient | formulation | wet mixture | OTF strip | OTF strip |
| Sodium alginate | 4.14% | 8.30 | 16.33% | 19.60 |
| MCT oil | 2.01% | 4.03 | 7.91% | 9.49 |
| Polysorbate 80 | 2.30% | 4.60 | 9.05% | 10.86 |
| Green tea extract | 4.49% | 9.00 | 17.71% | 21.25 |
| Glycerin | 2.07% | 4.14 | 8.13% | 9.75 |
| THC oil (76% THC) | 1.95% | 3.90 | 7.67% | 9.21 |
| Glyceryl Monocaprylate C8 | 1.20% | 2.40 | 4.71% | 5.65 |
| Water | 76.0% | 152.18 | 5.47% | 6.56 |
| Vitamin B5 | 0.50% | 1.01 | 1.98% | 2.38 |
| Xanthan gum | 0.10% | 0.20 | 0.39% | 0.47 |
| Waxy maize starch | 0.37% | 0.75 | 1.47% | 1.77 |
| Vitamin B6 | 0.30% | 0.60 | 1.18% | 1.41 |
| Terpenes | 0.15% | 0.31 | 0.61% | 0.73 |
| Yerba mate extract | 0.10% | 0.20 | 0.39% | 0.47 |
| Vitamin B12 | 0.0015% | 0.0030 | 0.01% | 0.01 |
| NHDC | 0.03% | 0.06 | 0.12% | 0.14 |
| Blend of Flavorants | 4.29% | 8.58 | 16.87% | 20.23 |

### Example 2

Quantities and proportions of ingredients for the following OTF formulation are described in Table 4 below.

Decarboxylated Cannabis extract was heated in an oven set at 60° C. until the fluid became flowable. Heated Cannabis extract was added to a pre-blended and pre-weighed oil phase mixture including MCTs, monoglycerides and diglycerides, terpenes, CBD extract and polysorbate 80 according to the quantities described in Table 4. The combined oil phase mixtures and Cannabis extract were mixed at moderate speed until the Cannabis extract was completely dispersed into the oil phase mixture. As soon as the Cannabis extract was completely dispersed in the oil phase mixture, heating was ceased and mixing was allowed to continue at approximately 500 rpm as the mixture cooled to room temperature.

Concurrently the aqueous phase was prepared. Water was added to a mixing vessel and mixing speed was set to 500 rpm at room temperature. A blend of active ingredients including valerian root extract, lavender extract, and L-theanine were added to the aqueous mixture according to the quantities described in Table 4 and mixing speed was increased to 600 rpm. A pre-weighed blend of flavorants, plasticizers, and other aqueous phase components including vegetable glycerin and modified waxy maize starch was added to the aqueous phase according to the quantities described in Table 4 and mixing continued at a speed of 600 rpm. Mixing of the aqueous phase including active ingredients continued at 600 rpm for 30 minutes. Mixing speed was reduced to 500 rpm and a mixture of sodium-alginate and xanthan gum was weighed according to the quantities described in Table 4. 50% of the sodium-alginate and xanthan gum mixture was added to the aqueous phase mixture and allowed to completely dissolve. Mixing speed was increased to 800 rpm and the remaining sodium-alginate and xanthan gum mixture was added and mixing continued at 500 rpm for 10 minutes. Mixing speed was increased to 1500 rpm to 2500 rpm and mixing of the aqueous phase continued for an additional 25 minutes. The blended oil phase was added to the aqueous phase while mixing continued at 1500 rpm to 2500 rpm for an additional 25 minutes to produce a homogenous emulsified film mixture.

A film casting knife was affixed to a substrate and set to a thickness of 550 μM. The emulsion mixture was poured into the reservoir of the film casting knife and manually spread across the surface of the substrate. The film casting knife was drawn across the pour emulsion mixture to produce a uniform layer across the substrate and excess overflow emulsion mixture was collected in an additional vessel.

A commercial dehydrator was pre-heated to a temperature of 37.5° C. The substrate layered with the emulsion mixture was placed in the 37.5° C. pre-heated dehydrator and allowed to dry 1 hour, after which point the temperature was lowered to 29.4° C. for an additional 2 hours and 20 minutes, until reaching a final moisture concentration of about 5.5±1% water. The dehydrated sheet was cut into individual 1.2″×0.75″ strips using conventional laser cutting equipment.

The dehydrated sheet was cut into individual 1.2″×0.75″ strips using conventional laser cutting equipment.

TABLE 4

| | % w/w in | Mg in a | % w/w in | Mg per |
|---|---|---|---|---|
| | mixed wet | 200 mg | dried final | dried final |
| Ingredient | formulation | wet mixture | OTF strip | OTF strip |
| Sodium alginate | 4.15% | 8.30 | 18.48% | 22.18 |
| Polysorbate 80 | 2.75% | 5.50 | 12.25% | 14.70 |
| Glycerin | 2.00% | 4.00 | 8.91% | 10.69 |
| MCT oil | 1.25% | 2.50 | 5.57% | 6.68 |
| Water | 78.78% | 157.57 | 5.49% | 6.59 |
| THC oil (76% THC) | 1.06% | 2.12 | 4.72% | 5.66 |
| Valerian root extract | 1.20% | 2.40 | 5.35% | 6.42 |
| CBD oil (70% CBD, 5% THC) | 1.36% | 2.72 | 6.06% | 7.27 |
| Waxy maize starch | 1.00% | 2.00 | 4.46% | 5.35 |
| L-theanine | 0.75% | 1.50 | 3.34% | 4.01 |
| Glyceryl Monocaprylate C8 | 0.70% | 1.40 | 3.12% | 3.74 |
| Lavender extract | 2.55% | 5.10 | 11.36% | 13.63 |
| Xanthan gum | 0.10% | 0.20 | 0.45% | 0.53 |
| Terpenes | 0.16% | 0.31 | 0.69% | 0.83 |
| Blend of Flavorants | 2.19% | 4.38 | 9.75% | 11.71 |

### Example 3

Quantities and proportions of ingredients for the following OTF formulation are described in Table 5 below.

Decarboxylated Cannabis extract was heated in an oven set at 60° C. until the fluid became flowable. Heated Cannabis extract was added to a pre-blended and pre-weighed oil phase mixture including MCTs, monoglycerides and diglycerides, vitamin A, vitamin E, terpenes, and polysorbate 80 according to the quantities described in Table 5. The combined oil phase mixtures and Cannabis extract were mixed at moderate speed until the Cannabis extract was completely dispersed into the oil phase mixture. As soon as the Cannabis extract was completely dispersed in the oil phase mixture, heating was ceased, and mixing was allowed to continue at approximately 500 rpm as the mixture cooled to room temperature.

Concurrently the aqueous phase was prepared. Water was added to a mixing vessel and mixing speed was set to 500 rpm at room temperature. A pre-weighed blend of flavorants, plasticizers, and other aqueous phase components including vegetable glycerin, modified waxy maize starch, vitamin B complex, L-theanine, Ginkgo biloba extract, and ginseng extract, was added to the aqueous phase according to the quantities described in Table 5 and mixing continued at a speed of 600 rpm. Mixing of the aqueous phase including active ingredients continued at 600 rpm for 30 minutes. Mixing speed was reduced to 500 rpm and sodium-alginate was weighed and added to the aqueous phase according to the quantities described in Table 5. 50% of the sodium-alginate was added to the aqueous phase mixture and allowed to completely dissolve. Mixing speed was increased to 800 rpm and the remaining sodium-alginate was added and mixing continued at 800 rpm for 10 minutes. Mixing speed was increased to 1500 rpm to 2500 rpm and mixing of the aqueous phase continued for an additional 25 minutes. The blended oil phase was added to the aqueous phase while mixing continued at 1500 rpm to 2500 rpm for an additional 25 minutes to produce a homogenous emulsified film mixture.

A film casting knife was affixed to a substrate and set to a thickness of 550 μM. The emulsion mixture was poured into the reservoir of the film casting knife and manually spread across the surface of the substrate. The film casting knife was drawn across the pour emulsion mixture to produce a uniform layer across the substrate and excess overflow emulsion mixture was collected in an additional vessel.

A commercial dehydrator was pre-heated to a temperature of 37.5° C. The substrate layered with the emulsion mixture was placed in the 37.5° C. pre-heated dehydrator and allowed to dry 1 hour, after which point the temperature was lowered to 29.4° C. for an additional 1 hours and 50 minutes, until reaching a final moisture concentration of about 5.5±1% water. The dehydrated sheet was cut into individual 1.2″×0.75″ strips using conventional laser cutting equipment.

TABLE 5

| | % w/w in | Mg in a | % w/w in | Mg per |
|---|---|---|---|---|
| | mixed wet | 200 mg | dried final | dried final |
| Ingredient | formulation | wet mixture | OTF strip | OTF strip |
| Sodium Alginate | 3.74% | 19.43 | 15.34% | 18.41 |
| Polysorbate 80 | 2.29% | 11.92 | 9.39% | 11.27 |
| Glycerin | 2.24% | 11.66 | 9.19% | 11.03 |
| THC oil (76% THC) | 2.39% | 12.41 | 9.80% | 11.76 |
| MCT oil | 2.19% | 11.40 | 9.11% | 10.93 |
| Water | 77.02% | 400.28 | 5.53% | 6.63 |
| Glyceryl Monocaprylate C8 | 0.88% | 4.56 | 3.64% | 4.37 |
| Waxy maize starch | 0.75% | 3.89 | 3.10% | 3.72 |
| Vitamin B12 | 0.09% | 0.47 | 0.37% | 0.44 |
| Vitamin B6 | 0.25% | 1.30 | 1.02% | 1.23 |
| Vitamin B9 | 0.02% | 0.08 | 0.04% | 0.05 |
| Vitamin A | 0.09% | 0.47 | 0.37% | 0.44 |
| Vitamin E | 0.75% | 3.91 | 3.07% | 3.69 |
| Ginkgo biloba extract | 1.07% | 5.54 | 4.39% | 5.27 |
| L-Theanine | 1.50% | 7.77 | 6.15% | 7.38 |
| Ginseng extract | 2.59% | 13.47 | 10.62% | 12.75 |
| Terpenes | 0.05% | 0.26 | 0.21% | 0.25 |
| Blend of Flavorants | 2.09% | 10.82 | 8.66% | 10.39 |

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1. A method of manufacturing an oral dissolvable thin film, comprising:
at least about 40% by weight of active ingredients, comprising:
a therapeutically effective amount of a first active ingredient that is a cannabinoid,
a therapeutically effective amount of a second active ingredient that is a botanical extract selected from the group consisting of Ginkgo biloba extract, ginseng extract, lavender extract, and valerian root extract, and
a therapeutically effective amount of a third active ingredient that is a vitamin selected from the group consisting of vitamin B5, vitamin B6, vitamin B9, vitamin B12, vitamin A, vitamin C, and vitamin E; and,
less than about 30% by weight of a film forming agent that is 2 or 3 ingredients selected from the group consisting of sodium alginate, modified waxy maize starch, and xanthan gum;
said method comprising:
combining the first active ingredient, second active ingredient, third active ingredient, and the film forming agent to form a stable homogenous dispersion;
casting the stable dispersion on a substrate into a sheet having a thickness less than about 800 μm;
drying the dispersion on the substrate at one or more temperatures less than 50° C. for a total duration of at least one hour to produce the oral dissolvable thin film; and
forming the dried film into strips adapted for use sublingually.

2-9. (canceled)

10. The method of claim 1, wherein forming comprises laser cutting the dried film into strips each having a mass less than 120 mg and thickness less than about 500 μm.

11. The method of claim 1, wherein the first active ingredient is one or more of THC, THCA, CBD, CBDA, or CBN.

12. The method of claim 1, wherein the second active ingredient comprises valerian root extract, lavender extract, L-theanine, or L-tryptophan.

13-14. (canceled)

15. A method of manufacturing an oral dissolvable thin film, comprising:
at least about 40% by weight of active ingredients, comprising:
a therapeutically effective amount of a first active ingredient that is a cannabinoid, a therapeutically effective amount of a second active ingredient that comprises caffeine,
a therapeutically effective amount of a third active ingredient that is a botanical extract selected from the group consisting of green tea extract and yerba mate extract, and
a therapeutically effective amount of a fourth active ingredient that is a vitamin selected from the group consisting of vitamin B5, vitamin B6, vitamin B9, vitamin B12, vitamin A, vitamin C, and vitamin E,
less than about 30% by weight of a film forming agent that is 2 or 3 ingredients selected from the group consisting of sodium alginate, modified waxy maize starch, and xanthan gum;
said method comprising:
combining the first active ingredient, second active ingredient, third active ingredient, fourth active ingredient, and the film forming agent to form a stable homogenous dispersion;
casting the stable dispersion on a substrate into a sheet having a thickness less than about 800 μm;
drying the dispersion on the substrate at one or more temperatures less than 50° C. for a total duration of at least one hour to produce the oral dissolvable thin film; and
forming the dried film into strips adapted for use sublingually.

16-24. (canceled)

25. The method of claim 15, wherein the forming comprises laser cutting the dried film into strips each having a mass less than 120 mg and thickness less than about 500 μm.

26. The method of claim 15, wherein the first active ingredient is one or more of THC, THCA, -CBD, CBDA, or CBN.

27-29. (canceled)

30. An oral dissolvable film composition manufactured utilizing the method of claim 15, having a percentage relative standard deviation of potency concentration of the at least one active ingredient in a batch of said oral dissolvable film composition of less than 1%.

31. The method of claim 1, wherein the film forming agent that consists essentially of 2 or 3 ingredients selected from the group consisting of sodium alginate, modified waxy maize starch, and xanthan gum.

32. The method of claim 1, wherein the first active ingredient is THC.

33. The method of claim 1, wherein the second active ingredient is valerian root extract.

34. The method of claim 1, wherein the third active ingredient is vitamin B12.

35. The method of claim 1, wherein the first active ingredient is THC, the second active ingredient is valerian root extract, and the third active ingredient is vitamin B12.

36. The method of claim 1, wherein the film forming agent is sodium alginate, modified waxy maize starch, and xanthan gum.

37. The method of claim 1, wherein the film forming agent is sodium alginate and xanthan gum.

38. The method of claim 1, wherein the film forming agent is sodium alginate and modified waxy maize starch.

39. The method of claim 1, wherein the film forming agent is xanthan gum and modified waxy maize starch.

40. The method of claim 1, wherein the film forming agent is sodium alginate present in an amount of 15-20% by weight of the thin film, modified waxy maize starch present in an amount of 1-5% by weight of the thin film, and xanthan gum present in an amount of 0.4-0.5% by weight of the thin film.

41. The method of claim 1, wherein the film forming agent is sodium alginate present in an amount of 15-20% by weight of the thin film and xanthan gum present in an amount 0.4-0.5% by weight of the thin film.

42. The method of claim 15, wherein the film forming agent consists essentially of 2 or 3 ingredients selected from the group consisting of sodium alginate, modified waxy maize starch, and xanthan gum.

43. The method of claim 15, wherein the first active ingredient is THC.

44. The method of claim 15, wherein the film forming agent is sodium alginate, modified waxy maize starch, and xanthan gum.

45. The method of claim 15, wherein the film forming agent is sodium alginate and xanthan gum.

46. The method of claim 15, wherein the film forming agent is sodium alginate and modified waxy maize starch.

47. The method of claim 15, wherein the film forming agent is xanthan gum and modified waxy maize starch.

48. The method of claim 15, wherein the film forming agent is sodium alginate present in an amount of 15-20% by weight of the thin film, modified waxy maize starch present in an amount of 1-5% by weight of the thin film, and xanthan gum present in an amount of 0.4-0.5% by weight of the thin film.

49. The method of claim 15, wherein the film forming agent is sodium alginate present in an amount of 15-20% by weight of the thin film and xanthan gum present in an amount 0.4-0.5% by weight of the thin film.
